(19) United States
(12) Patent Application Publication
(10) Pub. No.: US 2025/0002480 A1
(43) Pub. Date: Jan. 2, 2025

(54) AMINOHETEROARYL KINASE INHIBITORS

(71) Applicant: ALLORION THERAPEUTICS INC, Natick, MA (US)

(72) Inventors: Dai CHENG, Guangzhou (CN); Qiang DING, Guangzhou (CN); Tao ZHANG, Guangzhou (CN)

(21) Appl. No.: 18/670,469

(22) Filed: May 21, 2024

Related U.S. Application Data
(63) Continuation of application No. PCT/CN2022/133770, filed on Nov. 23, 2022.

(30) Foreign Application Priority Data
Nov. 26, 2021 (WO) PCT/CN2021/133429

(51) Int. Cl. C07D 405/12; A61K 31/635; C07D 239/47; C07D 309/10; C07D 309/22

(52) U.S. Cl. CPC C07D 405/12; A61K 31/635; C07D 239/47; C07D 309/10; C07D 309/22

(57) ABSTRACT

Provided herein are novel compounds, pharmaceutical compositions, and methods of using related to cyclin dependent kinases (CDKs). The compounds herein are typically CDK2 inhibitors, which can be used for treating a variety of diseases or disorders, such as cancer.

### CROSS REFERENCE TO RELATED APPLICATIONS

This Application is a continuation of International Application No. PCT/CN2022/133770, filed Nov. 23, 2022 which claims priority of International Application No. PCT/CN2021/133429, filed Nov. 26, 2021, the content of which are incorporated herein by reference in their entirety for all purposes.

### FIELD

In various embodiments, the present disclosure generally relates to novel heteroaryl compounds, compositions comprising the same, methods of preparing and methods of using the same, e.g., for inhibiting cyclin-dependent kinases and/or for treating or preventing various diseases or disorders described herein.

### BACKGROUND

Cyclin-dependent kinase (CDKs) are a family of serine/threonine protein kinases that regulate the cell cycle progression. Among CDKs, CDK2 is an essential driver for cells to transition from late G1 into S and G2 phases. During late G1, CDK2 is activated upon binding to cyclin E. The cyclin E/CDK2 complex hyper-phosphorylates RB to release E2F from Rb and initiate transcription of genes necessary for G1/S transition. Subsequently, CDK2 forms complex with Cyclin A to regulate S phase progression by activating proteins important for DNA replication and centrosome duplication, such as DNA replication licensing protein (CDC6) and centrosome protein CP110 (Tadesse et al. Targeting CDK2 in cancer: challenges and opportunities for therapy, Drug Discovery Today. 2019; 25(2): 406-413).

Cyclin E1 is frequently amplified and/or overexpressed in human cancer. In high grade serous ovarian cancer, cyclin E1 amplification is detected in approximately 20% of patients and is associated with chemo resistance/refractory (TCGA, Integrated genomic analyses of ovarian carcinoma, Nature. 2011; 474: 609-615; Nakayama et al; Gene amplification CCNE1 is related to poor survival and potential therapeutic target in ovarian cancer, Cancer (2010) 116: 2621-34). Cyclin E1 amplified ovarian cancer cell lines are sensitive to reagents that either inhibit CDK2 activity or decrease cellular CDK2 protein level, suggesting CDK2 dependence in these cyclin E1 amplified cells (Au-Yeung et al. Selective targeting of cyclin E1 amplified high grade serous ovarian cancer by clin-dependent kinase 2 and AKT inhibition, Clin. Cancer Res. 2017; 23(7):1862-1874). Poor outcomes and drug resistance were also associated with high Cyclin E1 expression in endometrial, gastric, breast and other cancers (Noske et al., Detection of CCNE1/URI (19q12) amplification by in situ hybridization is common in high grade and type II endometrial cancer, Oncotarget (2017) 8: 14794-14805; Ooi et al., Gene amplification of CCNE1, CCND1 and CDK6 in gastric cancers detected by multiplex ligation-dependent probe amplification and fluorescence in situ hybridization, Hum Pathol. (2017) 61:58-67; Keyomarsi et al., Cyclin E and survival in patients with breast cancer. N Engl J Med. (2002) 347: 1566-75). Estrogen receptor (ER) positive breast cancer cell lines with acquired resistance to CDK4/6 inhibitor Palbociclib has elevated cyclin E1 expression and can be re-sensitized upon knock down of CDK2 (Herrera-Abreu et al., Early adaptation and acquired resistance to CDK4/6 inhibition in estrogen receptor-positive breast cancer, Cancer Res. (2016) 76: 2301-2313). High cyclin E1 level was also reported to associate with poor response to Palbociclib plus fulvestrant combo therapy in ER+BC (CCNE1 high vs CCNE1 low: median PFS for Palbociclib+fulvestrant arm, 7.6 v 14.1 month; placebo+fulvestrant arm, 4.0 v 4.8 month) further underline the importance of CDK2 activity in mediating resistance to CDK4/6 inhibitors (Turner et al., Cyclin E1 expression and Palbociclib efficacy in previously treated hormone receptor positive metastatic breast cancer Clin Oncol. (2019) 37(14): 1169-1178).

Cyclin E2 (CCNE2) overexpression was reported as associated with endocrine resistance in breast cancer cells and CDK2 inhibition has been reported to restore sensitivity to tamoxifen or CDK4 inhibitors in tamoxifen-resistant and CCNE2 overexpressing cells. (Caldon et al., Cyclin E2 overexpression is associated with endocrine resistance but not insensitivity to CDK2 inhibition in human breast cancer cells. Mol Cancer Ther. (2012) 11:1488-99; Herrera-Abreu et al., Early Adaptation and Acquired Resistance to CDK4/6 Inhibition in Estrogen Receptor-Positive Breast Cancer, Cancer Res. (2016) 76: 2301-2313). Additionally, Cyclin E amplification has also been reported as contributing to trastuzumab resistance in HER2+ breast cancer. (Scaltriti et al. Cyclin E amplification/overexpression is a mechanism of trastuzumab resistance in HER2+ breast cancer patients, Proc Natl Acad Sci. (2011) 108: 3761-6). Further, Cyclin E overexpression was reported to play a role in basal-like and triple negative breast cancer (TNBC), as well as inflammatory breast cancer. (Elsawaf & Sinn, Triple Negative Breast Cancer: Clinical and Histological Correlations, Breast Care (2011) 6:273-278; Alexander et al., Cyclin E overexpression as a biomarker for combination treatment strategies in inflammatory breast cancer, Oncotarget (2017) 8: 14897-14911.)

### BRIEF SUMMARY

The importance of CDK2 in proliferative pathways and the frequently altered CDK2/cyclin E1 activity in tumor highlights CDK2 as a target for cancer treatment. CDK2 knock out mice are viable with minimum defects, suggesting CDK2 is not essential for normal cell proliferation (Berthet et al., CDK2 knock out mice are viable. Curr Biol. (2003) 13(20):1775-85). In addition, selective CDK2 inhibitors may minimize clinical toxicity while being active in treating patients with high tumor cyclinE1 and/or E2 expression. However, in some embodiments, inhibiting CDK2 as well as other CDKs can also be clinically beneficial.

In various embodiments, the present disclosure relates to novel heteroaryl compounds which can inhibit CDK2, e.g., selectively over other CDKs and/or other kinases. The compounds and compositions herein are useful for treating various diseases or disorders, such as cancer, e.g., those characterized with amplification or overexpression of Cyclin E1 (CCNE1) and/or cyclin E2 (CCNE2).

In some embodiments, the present disclosure provides specific compounds as disclosed in Table 1 herein, or a pharmaceutically acceptable salt thereof. In some embodiments, the present disclosure provides specific compounds according to E1 to E7 as described herein, or a pharmaceutically acceptable salt thereof.

In some embodiments, the present disclosure provides a pharmaceutical composition comprising one or more compounds of the present disclosure and optionally a pharmaceutically acceptable excipient. The pharmaceutical composition can be typically formulated for oral administration.

In some embodiments, the present disclosure also provides a method of inhibiting CDK activity such as CDK2 activity in a subject or biological sample. In some embodiments, the method comprises contacting the subject or biological sample with an effective amount of one or more compounds of the present disclosure, e.g., any of Examples E1-E7, or any of the specific compounds disclosed in Table 1 herein, or a pharmaceutically acceptable salt thereof, or a pharmaceutical composition comprising the same.

In some embodiments, the present disclosure provides a method of treating or preventing a CDK-mediated disease or disorder in a subject in need thereof. In some embodiments, the method comprises administering to the subject an effective amount of one or more compounds of the present disclosure or the pharmaceutical composition herein. In some embodiments, the method comprises administering to the subject an effective amount of any of Examples E1-E7, or any of the specific compounds disclosed in Table 1 herein, or a pharmaceutically acceptable salt thereof, or a pharmaceutical composition comprising the same.

In some embodiments, the present disclosure also provides a method of treating or preventing cancer in a subject in need thereof, which comprises administering to the subject an effective amount of a compound of the present disclosure (e.g., any of Examples E1-E7, or any of the specific compounds disclosed in Table 1 herein, or a pharmaceutically acceptable salt thereof) or an effective amount of a pharmaceutical composition described herein. In some embodiments, the cancer is characterized by amplification or overexpression of CCNE1 and/or CCNE2. In some embodiments, the cancer is selected from breast cancer, ovarian cancer, bladder cancer, uterine cancer, prostate cancer, lung cancer (including NSCLC, SCLC, squamous cell carcinoma or adenocarcinoma), esophageal cancer, head and neck cancer, colorectal cancer, kidney cancer (including RCC), liver cancer (including HCC), pancreatic cancer, stomach (i.e., gastric) cancer, thyroid cancer, and combinations thereof. In some embodiments, the cancer is breast cancer selected from ER-positive/HR-positive, HER2-negative breast cancer; ER-positive/HR-positive, HER2-positive breast cancer; triple negative breast cancer (TNBC); and inflammatory breast cancer. In some embodiments, the cancer is breast cancer. In some embodiments, the cancer is breast cancer selected from endocrine resistant breast cancer, trastuzumab resistant breast cancer, or breast cancer demonstrating primary or acquired resistance to CDK4/CDK6 inhibition. In some embodiments, the cancer is advanced or metastatic breast cancer. In some embodiments, the cancer is ovarian cancer.

The administering in the methods herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the administering is orally. In some embodiments, the administering is a parenteral injection, such as an intraveneous injection.

Compounds of the present disclosure can be used as a monotherapy or in a combination therapy. In some embodiments according to the methods described herein, one or more compounds of the present disclosure can be administered as the only active ingredient(s). In some embodiments, the method herein further comprises administering to the subject an additional therapeutic agent, such as additional anticancer agents described herein.

It is to be understood that both the foregoing summary and the following detailed description are exemplary and explanatory only, and are not restrictive of the invention herein.

### DETAILED DESCRIPTION

In various embodiments, the present disclosure provides compounds and compositions that are useful for inhibiting CDKs such as CDK2 and/or treating or preventing various diseases or disorders described herein, e.g., cancer.

The compounds herein can typically inhibit CDK2. In some embodiments, the compounds herein can selectively inhibit CDK2 over other CDKs. In some specific embodiments, the present disclosure provide a compound selected from Table 1 below, a deuterated analog thereof, a stereoisomer thereof, or a pharmaceutically acceptable salt thereof:

TABLE 1
List of Compounds

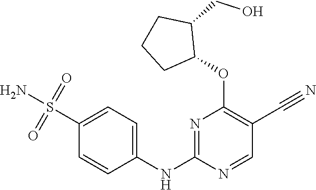 [SMILES: N#Cc1cnc(Nc2ccc(S(N)(=O)=O)cc2)nc1O[C@@H]1CCC[C@@H]1CO]

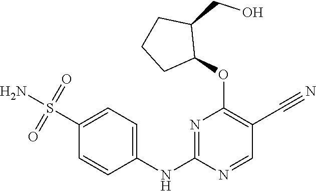 [SMILES: N#Cc1cnc(Nc2ccc(S(N)(=O)=O)cc2)nc1O[C@H]1CCC[C@H]1CO]

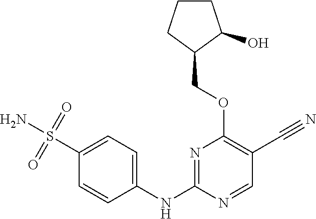 [SMILES: N#Cc1cnc(Nc2ccc(S(N)(=O)=O)cc2)nc1OC[C@H]1CCC[C@H]1O]

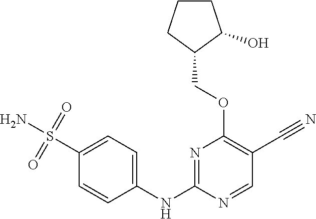 [SMILES: N#Cc1cnc(Nc2ccc(S(N)(=O)=O)cc2)nc1OC[C@@H]1CCC[C@@H]1O]

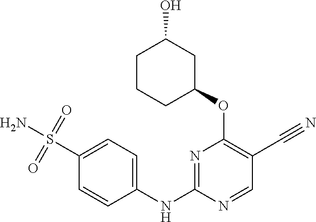 [SMILES: N#Cc1cnc(Nc2ccc(S(N)(=O)=O)cc2)nc1O[C@H]1CCC[C@H](O)C1]

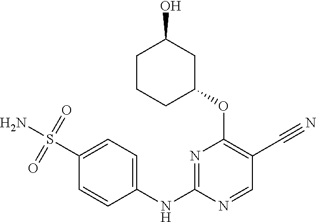 [SMILES: N#Cc1cnc(Nc2ccc(S(N)(=O)=O)cc2)nc1O[C@@H]1CCC[C@@H](O)C1]

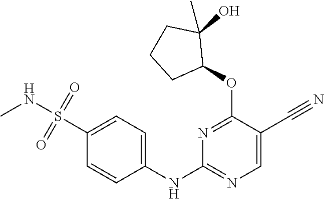 [SMILES: CNS(=O)(=O)c1ccc(Nc2ncc(C#N)c(O[C@H]3CCC[C@@]3(C)O)n2)cc1]

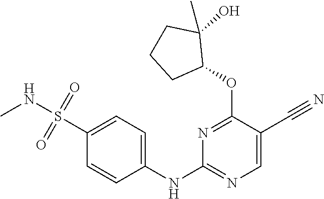 [SMILES: CNS(=O)(=O)c1ccc(Nc2ncc(C#N)c(O[C@@H]3CCC[C@]3(C)O)n2)cc1]

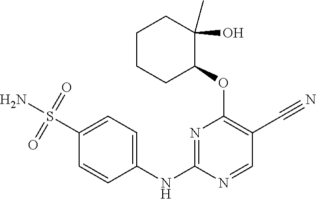 [SMILES: C[C@@]1(O)CCCC[C@@H]1Oc1nc(Nc2ccc(S(N)(=O)=O)cc2)ncc1C#N]

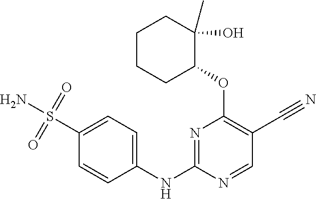 [SMILES: C[C@]1(O)CCCC[C@H]1Oc1nc(Nc2ccc(S(N)(=O)=O)cc2)ncc1C#N]

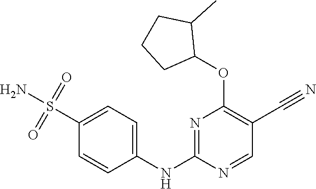 [SMILES: CC1CCCC1Oc1nc(Nc2ccc(S(N)(=O)=O)cc2)ncc1C#N]

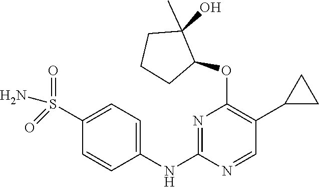 [SMILES: C[C@@]1(O)CCC[C@@H]1Oc1nc(Nc2ccc(S(N)(=O)=O)cc2)ncc1C1CC1]

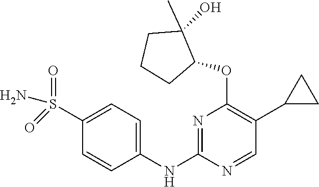 [SMILES: C[C@]1(O)CCC[C@H]1Oc1nc(Nc2ccc(S(N)(=O)=O)cc2)ncc1C1CC1]

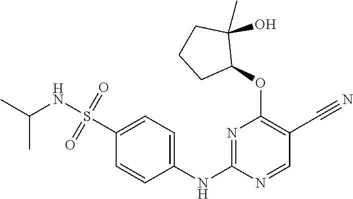 [SMILES: CC(C)NS(=O)(=O)c1ccc(Nc2ncc(C#N)c(O[C@H]3CCC[C@@]3(C)O)n2)cc1]

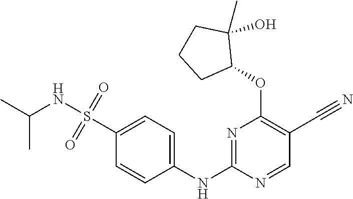 [SMILES: CC(C)NS(=O)(=O)c1ccc(Nc2ncc(C#N)c(O[C@@H]3CCC[C@]3(C)O)n2)cc1]

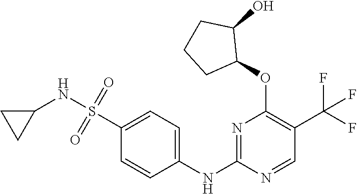 [SMILES: O=S(=O)(NC1CC1)c1ccc(Nc2ncc(C(F)(F)F)c(O[C@H]3CCC[C@H]3O)n2)cc1]

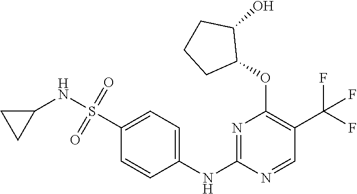 [SMILES: O=S(=O)(NC1CC1)c1ccc(Nc2ncc(C(F)(F)F)c(O[C@@H]3CCC[C@@H]3O)n2)cc1]

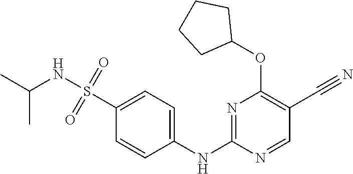 [SMILES: CC(C)NS(=O)(=O)c1ccc(Nc2ncc(C#N)c(OC3CCCC3)n2)cc1]

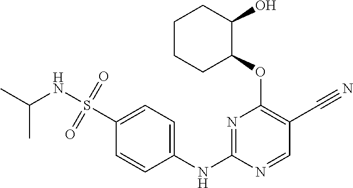 [SMILES: CC(C)NS(=O)(=O)c1ccc(Nc2ncc(C#N)c(O[C@H]3CCCC[C@H]3O)n2)cc1]

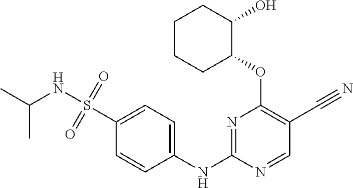 [SMILES: CC(C)NS(=O)(=O)c1ccc(Nc2ncc(C#N)c(O[C@@H]3CCCC[C@@H]3O)n2)cc1]

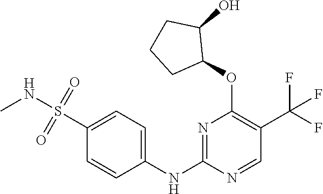 [SMILES: CNS(=O)(=O)c1ccc(Nc2ncc(C(F)(F)F)c(O[C@H]3CCC[C@H]3O)n2)cc1]

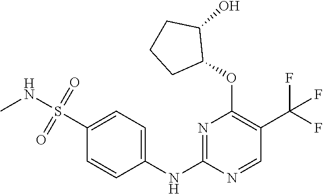 [SMILES: CNS(=O)(=O)c1ccc(Nc2ncc(C(F)(F)F)c(O[C@@H]3CCC[C@@H]3O)n2)cc1]

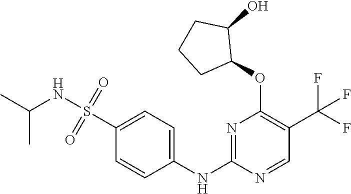 [SMILES: CC(C)NS(=O)(=O)c1ccc(Nc2ncc(C(F)(F)F)c(O[C@H]3CCC[C@H]3O)n2)cc1]

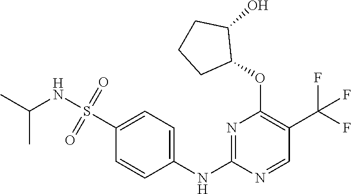 [SMILES: CC(C)NS(=O)(=O)c1ccc(Nc2ncc(C(F)(F)F)c(O[C@@H]3CCC[C@@H]3O)n2)cc1]

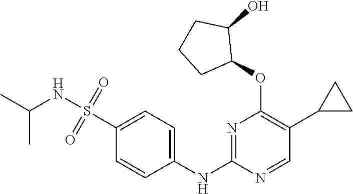 [SMILES: CC(C)NS(=O)(=O)c1ccc(Nc2ncc(C3CC3)c(O[C@H]3CCC[C@H]3O)n2)cc1]

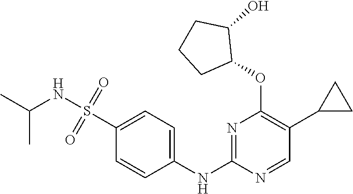 [SMILES: CC(C)NS(=O)(=O)c1ccc(Nc2ncc(C3CC3)c(O[C@@H]3CCC[C@@H]3O)n2)cc1]

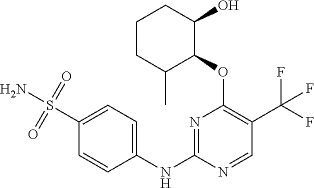 [SMILES: CC1CCC[C@@H](O)[C@H]1Oc1nc(Nc2ccc(S(N)(=O)=O)cc2)ncc1C(F)(F)F]

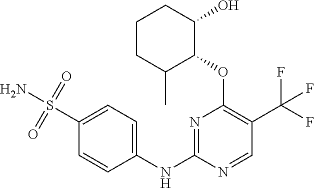 [SMILES: CC1CCC[C@H](O)[C@@H]1Oc1nc(Nc2ccc(S(N)(=O)=O)cc2)ncc1C(F)(F)F]

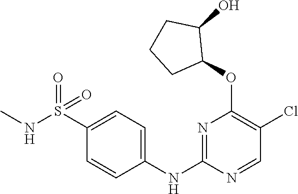 [SMILES: CNS(=O)(=O)c1ccc(Nc2ncc(Cl)c(O[C@H]3CCC[C@H]3O)n2)cc1]

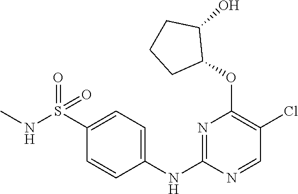 [SMILES: CNS(=O)(=O)c1ccc(Nc2ncc(Cl)c(O[C@@H]3CCC[C@@H]3O)n2)cc1]

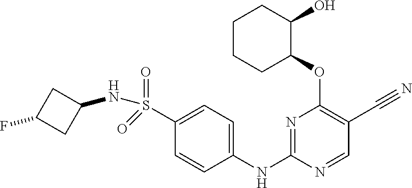

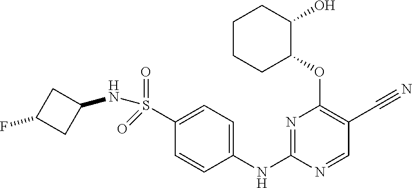

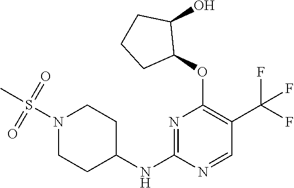 [SMILES: CS(=O)(=O)N1CCC(Nc2ncc(C(F)(F)F)c(O[C@H]3CCC[C@H]3O)n2)CC1]

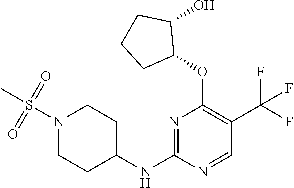 [SMILES: CS(=O)(=O)N1CCC(Nc2ncc(C(F)(F)F)c(O[C@@H]3CCC[C@@H]3O)n2)CC1]

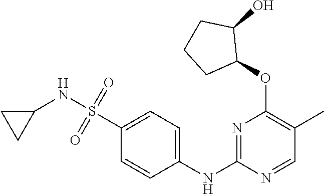 [SMILES: Cc1cnc(Nc2ccc(S(=O)(=O)NC3CC3)cc2)nc1O[C@H]1CCC[C@H]1O]

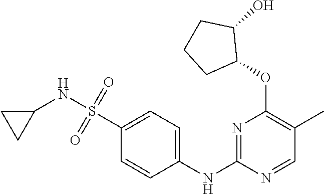 [SMILES: Cc1cnc(Nc2ccc(S(=O)(=O)NC3CC3)cc2)nc1O[C@@H]1CCC[C@@H]1O]

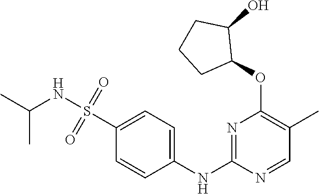 [SMILES: Cc1cnc(Nc2ccc(S(=O)(=O)NC(C)C)cc2)nc1O[C@H]1CCC[C@H]1O]

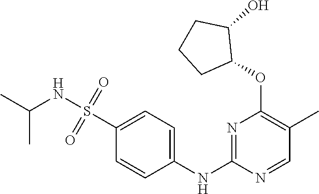 [SMILES: Cc1cnc(Nc2ccc(S(=O)(=O)NC(C)C)cc2)nc1O[C@@H]1CCC[C@@H]1O]

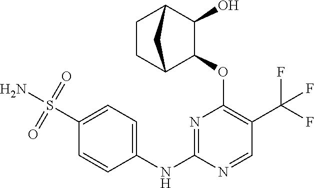 [SMILES: NS(=O)(=O)c1ccc(Nc2ncc(C(F)(F)F)c(O[C@H]3[C@@H]4CC[C@@H](C4)[C@H]3O)n2)cc1]

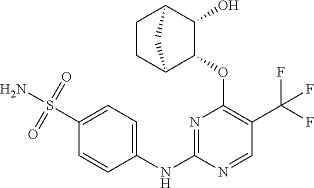 [SMILES: NS(=O)(=O)c1ccc(Nc2ncc(C(F)(F)F)c(O[C@@H]3[C@H]4CC[C@H](C4)[C@@H]3O)n2)cc1]

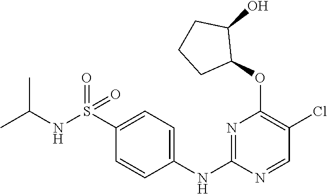 [SMILES: CC(C)NS(=O)(=O)c1ccc(Nc2ncc(Cl)c(O[C@H]3CCC[C@H]3O)n2)cc1]

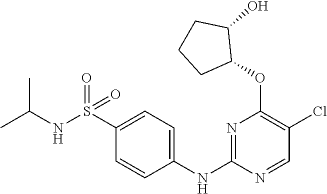 [SMILES: CC(C)NS(=O)(=O)c1ccc(Nc2ncc(Cl)c(O[C@@H]3CCC[C@@H]3O)n2)cc1]

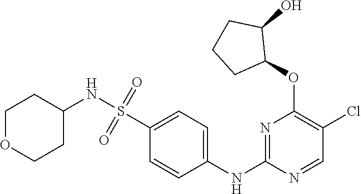 [SMILES: O=S(=O)(NC1CCOCC1)c1ccc(Nc2ncc(Cl)c(O[C@H]3CCC[C@H]3O)n2)cc1]

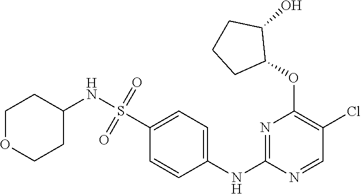 [SMILES: O=S(=O)(NC1CCOCC1)c1ccc(Nc2ncc(Cl)c(O[C@@H]3CCC[C@@H]3O)n2)cc1]

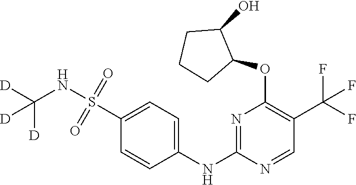 [SMILES: [2H]C([2H])([2H])NS(=O)(=O)c1ccc(Nc2ncc(C(F)(F)F)c(O[C@H]3CCC[C@H]3O)n2)cc1]

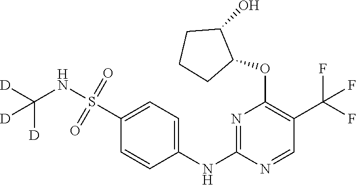 [SMILES: [2H]C([2H])([2H])NS(=O)(=O)c1ccc(Nc2ncc(C(F)(F)F)c(O[C@@H]3CCC[C@@H]3O)n2)cc1]

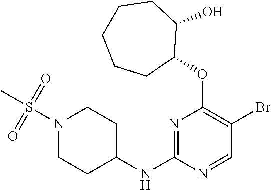 [SMILES: CS(=O)(=O)N1CCC(Nc2ncc(Br)c(O[C@@H]3CCCCC[C@@H]3O)n2)CC1]

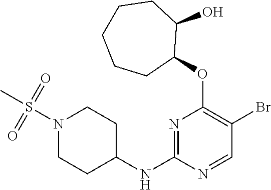 [SMILES: CS(=O)(=O)N1CCC(Nc2ncc(Br)c(O[C@H]3CCCCC[C@H]3O)n2)CC1]

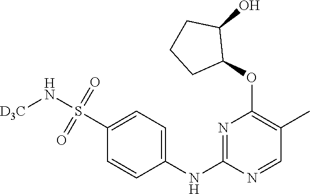 [SMILES: [2H]C([2H])([2H])NS(=O)(=O)c1ccc(Nc2ncc(C)c(O[C@H]3CCC[C@H]3O)n2)cc1]

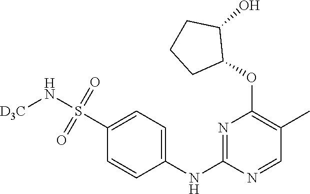 [SMILES: [2H]C([2H])([2H])NS(=O)(=O)c1ccc(Nc2ncc(C)c(O[C@@H]3CCC[C@@H]3O)n2)cc1]

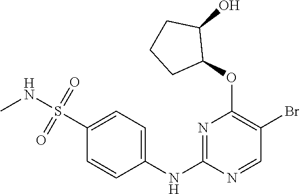 [SMILES: CNS(=O)(=O)c1ccc(Nc2ncc(Br)c(O[C@H]3CCC[C@H]3O)n2)cc1]

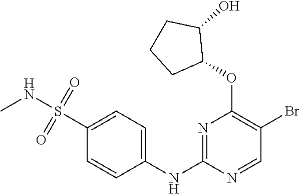 [SMILES: CNS(=O)(=O)c1ccc(Nc2ncc(Br)c(O[C@@H]3CCC[C@@H]3O)n2)cc1]

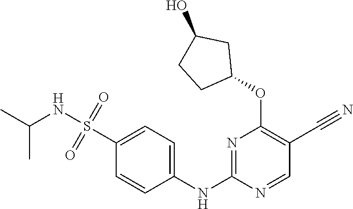 [SMILES: CC(C)NS(=O)(=O)c1ccc(Nc2ncc(C#N)c(O[C@@H]3CC[C@@H](O)C3)n2)cc1]

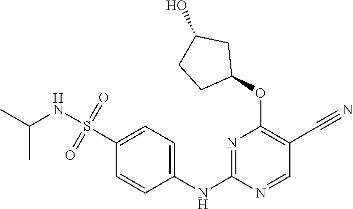 [SMILES: CC(C)NS(=O)(=O)c1ccc(Nc2ncc(C#N)c(O[C@H]3CC[C@H](O)C3)n2)cc1]

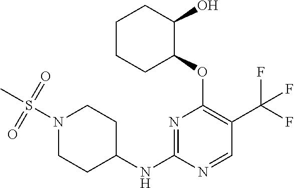 [SMILES: CS(=O)(=O)N1CCC(Nc2ncc(C(F)(F)F)c(O[C@H]3CCCC[C@H]3O)n2)CC1]

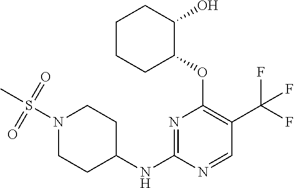 [SMILES: CS(=O)(=O)N1CCC(Nc2ncc(C(F)(F)F)c(O[C@@H]3CCCC[C@@H]3O)n2)CC1]

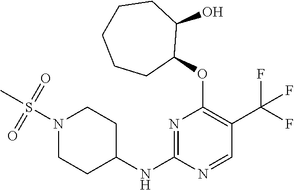 [SMILES: CS(=O)(=O)N1CCC(Nc2ncc(C(F)(F)F)c(O[C@H]3CCCCC[C@H]3O)n2)CC1]

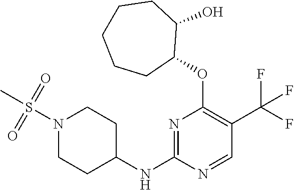 [SMILES: CS(=O)(=O)N1CCC(Nc2ncc(C(F)(F)F)c(O[C@@H]3CCCCC[C@@H]3O)n2)CC1]

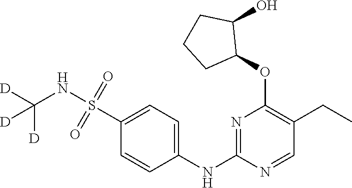 [SMILES: [2H]C([2H])([2H])NS(=O)(=O)c1ccc(Nc2ncc(CC)c(O[C@H]3CCC[C@H]3O)n2)cc1]

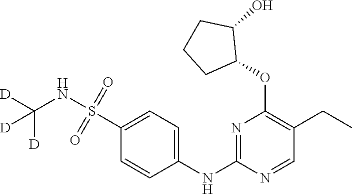 [SMILES: [2H]C([2H])([2H])NS(=O)(=O)c1ccc(Nc2ncc(CC)c(O[C@@H]3CCC[C@@H]3O)n2)cc1]

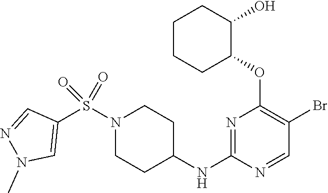 [SMILES: Cn1cc(S(=O)(=O)N2CCC(Nc3ncc(Br)c(O[C@@H]4CCCC[C@@H]4O)n3)CC2)cn1]

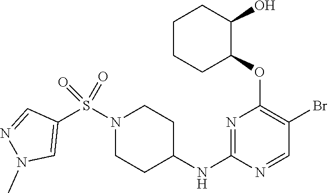 [SMILES: Cn1cc(S(=O)(=O)N2CCC(Nc3ncc(Br)c(O[C@H]4CCCC[C@H]4O)n3)CC2)cn1]

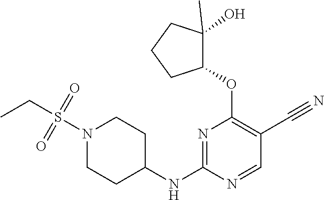 [SMILES: CCS(=O)(=O)N1CCC(Nc2ncc(C#N)c(O[C@@H]3CCC[C@]3(C)O)n2)CC1]

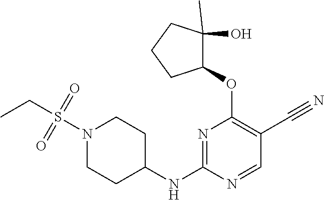 [SMILES: CCS(=O)(=O)N1CCC(Nc2ncc(C#N)c(O[C@H]3CCC[C@@]3(C)O)n2)CC1]

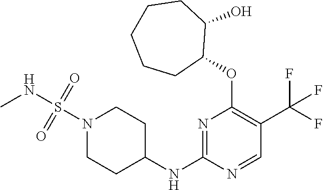 [SMILES: CNS(=O)(=O)N1CCC(Nc2ncc(C(F)(F)F)c(O[C@@H]3CCCCC[C@@H]3O)n2)CC1]

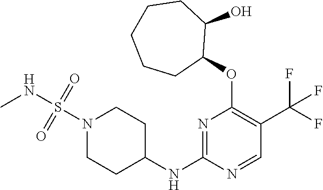 [SMILES: CNS(=O)(=O)N1CCC(Nc2ncc(C(F)(F)F)c(O[C@H]3CCCCC[C@H]3O)n2)CC1]

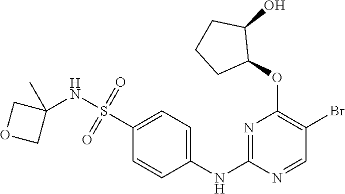 [SMILES: CC1(NS(=O)(=O)c2ccc(Nc3ncc(Br)c(O[C@H]4CCC[C@H]4O)n3)cc2)COC1]

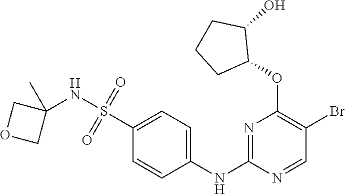 [SMILES: CC1(NS(=O)(=O)c2ccc(Nc3ncc(Br)c(O[C@@H]4CCC[C@@H]4O)n3)cc2)COC1]

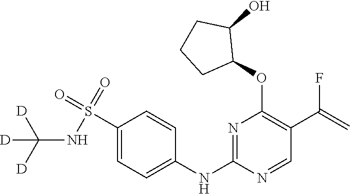 [SMILES: [2H]C([2H])([2H])NS(=O)(=O)c1ccc(Nc2ncc(C(=C)F)c(O[C@H]3CCC[C@H]3O)n2)cc1]

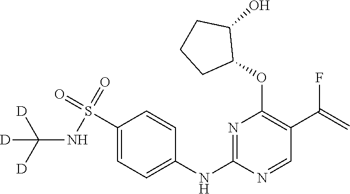 [SMILES: [2H]C([2H])([2H])NS(=O)(=O)c1ccc(Nc2ncc(C(=C)F)c(O[C@@H]3CCC[C@@H]3O)n2)cc1]

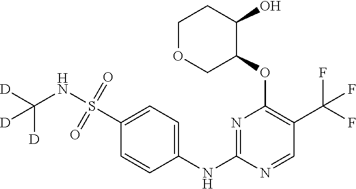 [SMILES: [2H]C([2H])([2H])NS(=O)(=O)c1ccc(Nc2ncc(C(F)(F)F)c(O[C@H]3COCC[C@H]3O)n2)cc1]

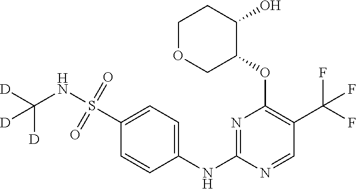 [SMILES: [2H]C([2H])([2H])NS(=O)(=O)c1ccc(Nc2ncc(C(F)(F)F)c(O[C@@H]3COCC[C@@H]3O)n2)cc1]

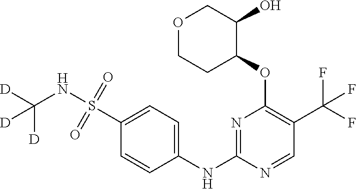 [SMILES: [2H]C([2H])([2H])NS(=O)(=O)c1ccc(Nc2ncc(C(F)(F)F)c(O[C@H]3CCOC[C@H]3O)n2)cc1]

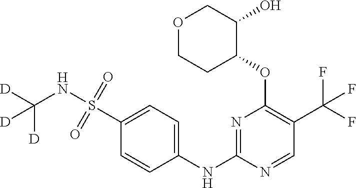 [SMILES: [2H]C([2H])([2H])NS(=O)(=O)c1ccc(Nc2ncc(C(F)(F)F)c(O[C@@H]3CCOC[C@@H]3O)n2)cc1]

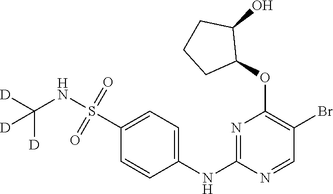 [SMILES: [2H]C([2H])([2H])NS(=O)(=O)c1ccc(Nc2ncc(Br)c(O[C@H]3CCC[C@H]3O)n2)cc1]

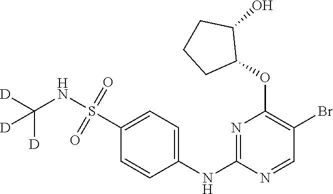 [SMILES: [2H]C([2H])([2H])NS(=O)(=O)c1ccc(Nc2ncc(Br)c(O[C@@H]3CCC[C@@H]3O)n2)cc1]

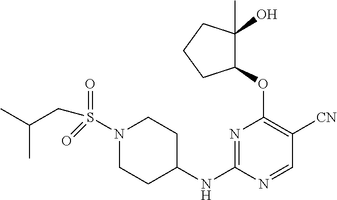 [SMILES: CC(C)CS(=O)(=O)N1CCC(Nc2ncc(C#N)c(O[C@H]3CCC[C@@]3(C)O)n2)CC1]

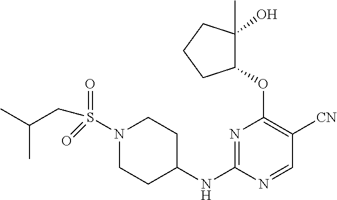 [SMILES: CC(C)CS(=O)(=O)N1CCC(Nc2ncc(C#N)c(O[C@@H]3CCC[C@]3(C)O)n2)CC1]

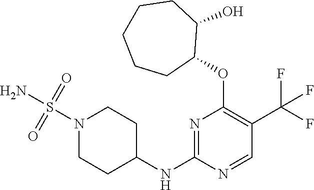 [SMILES: NS(=O)(=O)N1CCC(Nc2ncc(C(F)(F)F)c(O[C@@H]3CCCCC[C@@H]3O)n2)CC1]

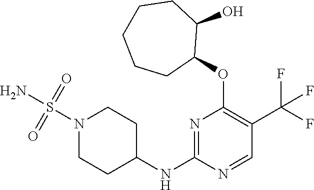 [SMILES: NS(=O)(=O)N1CCC(Nc2ncc(C(F)(F)F)c(O[C@H]3CCCCC[C@H]3O)n2)CC1]

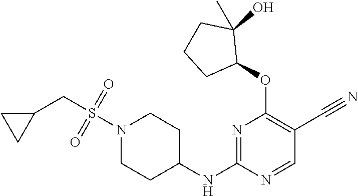 [SMILES: C[C@@]1(O)CCC[C@@H]1Oc1nc(NC2CCN(S(=O)(=O)CC3CC3)CC2)ncc1C#N]

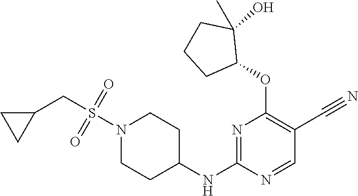 [SMILES: C[C@]1(O)CCC[C@H]1Oc1nc(NC2CCN(S(=O)(=O)CC3CC3)CC2)ncc1C#N]

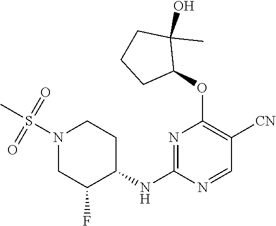 [SMILES: C[C@@]1(O)CCC[C@@H]1Oc1nc(N[C@H]2CCN(S(C)(=O)=O)C[C@H]2F)ncc1C#N]

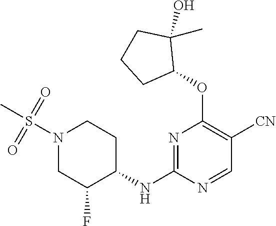 [SMILES: C[C@]1(O)CCC[C@H]1Oc1nc(N[C@H]2CCN(S(C)(=O)=O)C[C@H]2F)ncc1C#N]

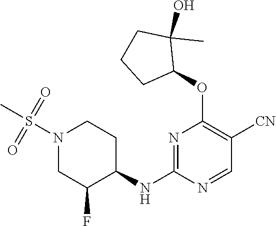 [SMILES: C[C@@]1(O)CCC[C@@H]1Oc1nc(N[C@@H]2CCN(S(C)(=O)=O)C[C@@H]2F)ncc1C#N]

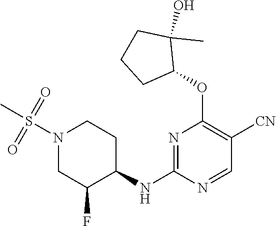 [SMILES: C[C@]1(O)CCC[C@H]1Oc1nc(N[C@@H]2CCN(S(C)(=O)=O)C[C@@H]2F)ncc1C#N]

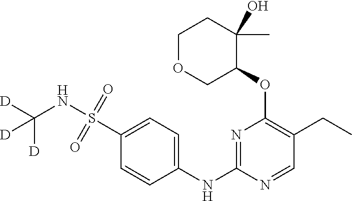 [SMILES: [2H]C([2H])([2H])NS(=O)(=O)c1ccc(Nc2ncc(CC)c(O[C@H]3COCC[C@@]3(C)O)n2)cc1]

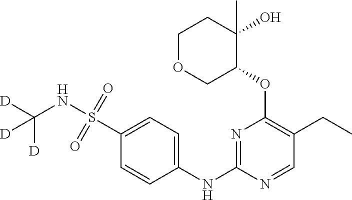 [SMILES: [2H]C([2H])([2H])NS(=O)(=O)c1ccc(Nc2ncc(CC)c(O[C@@H]3COCC[C@]3(C)O)n2)cc1]

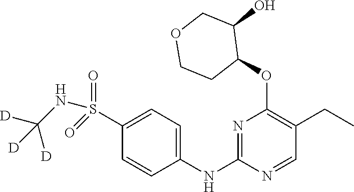 [SMILES: [2H]C([2H])([2H])NS(=O)(=O)c1ccc(Nc2ncc(CC)c(O[C@H]3CCOC[C@H]3O)n2)cc1]

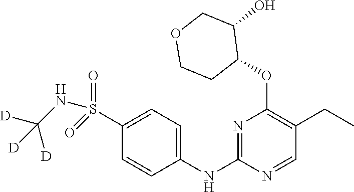 [SMILES: [2H]C([2H])([2H])NS(=O)(=O)c1ccc(Nc2ncc(CC)c(O[C@@H]3CCOC[C@@H]3O)n2)cc1]

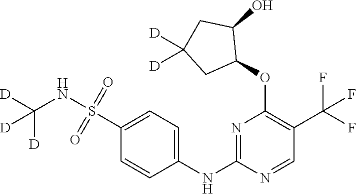 [SMILES: [2H]C1([2H])C[C@@H](O)[C@@H](Oc2nc(Nc3ccc(S(=O)(=O)NC([2H])([2H])[2H])cc3)ncc2C(F)(F)F)C1]

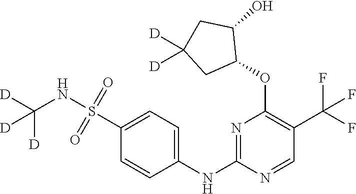 [SMILES: [2H]C1([2H])C[C@H](O)[C@H](Oc2nc(Nc3ccc(S(=O)(=O)NC([2H])([2H])[2H])cc3)ncc2C(F)(F)F)C1]

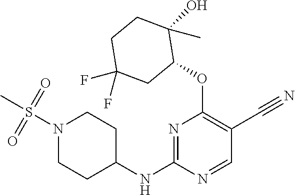 [SMILES: C[C@]1(O)CCC(F)(F)C[C@H]1Oc1nc(NC2CCN(S(C)(=O)=O)CC2)ncc1C#N]

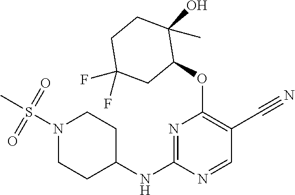 [SMILES: C[C@@]1(O)CCC(F)(F)C[C@@H]1Oc1nc(NC2CCN(S(C)(=O)=O)CC2)ncc1C#N]

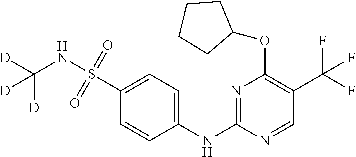 [SMILES: [2H]C([2H])([2H])NS(=O)(=O)c1ccc(Nc2ncc(C(F)(F)F)c(OC3CCCC3)n2)cc1]

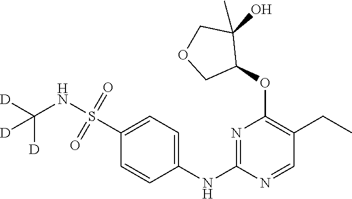 [SMILES: [2H]C([2H])([2H])NS(=O)(=O)c1ccc(Nc2ncc(CC)c(O[C@H]3COC[C@@]3(C)O)n2)cc1]

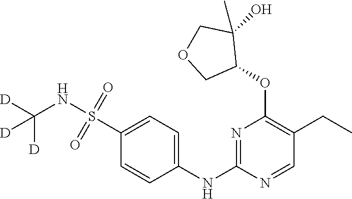 [SMILES: [2H]C([2H])([2H])NS(=O)(=O)c1ccc(Nc2ncc(CC)c(O[C@@H]3COC[C@]3(C)O)n2)cc1]

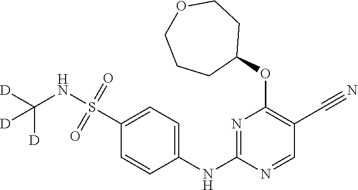 [SMILES: [2H]C([2H])([2H])NS(=O)(=O)c1ccc(Nc2ncc(C#N)c(O[C@H]3CCCOCC3)n2)cc1]

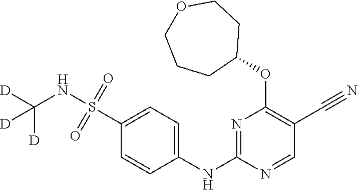 [SMILES: [2H]C([2H])([2H])NS(=O)(=O)c1ccc(Nc2ncc(C#N)c(O[C@@H]3CCCOCC3)n2)cc1]

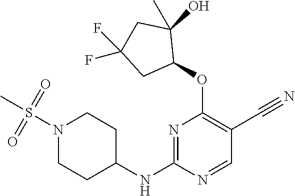 [SMILES: C[C@@]1(O)CC(F)(F)C[C@@H]1Oc1nc(NC2CCN(S(C)(=O)=O)CC2)ncc1C#N]

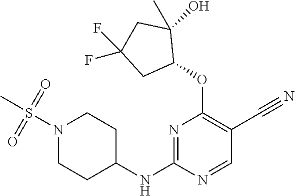 [SMILES: C[C@]1(O)CC(F)(F)C[C@H]1Oc1nc(NC2CCN(S(C)(=O)=O)CC2)ncc1C#N]

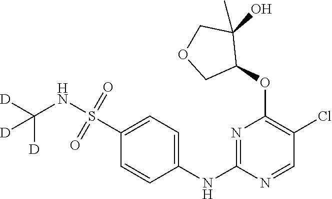 [SMILES: [2H]C([2H])([2H])NS(=O)(=O)c1ccc(Nc2ncc(Cl)c(O[C@H]3COC[C@@]3(C)O)n2)cc1]

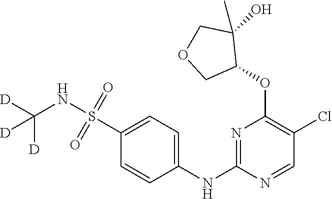 [SMILES: [2H]C([2H])([2H])NS(=O)(=O)c1ccc(Nc2ncc(Cl)c(O[C@@H]3COC[C@]3(C)O)n2)cc1]

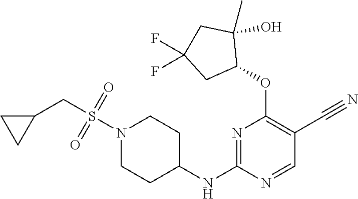 [SMILES: C[C@]1(O)CC(F)(F)C[C@H]1Oc1nc(NC2CCN(S(=O)(=O)CC3CC3)CC2)ncc1C#N]

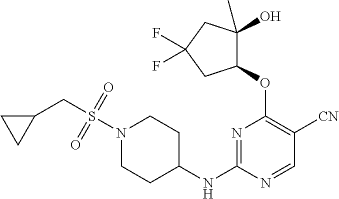 [SMILES: C[C@@]1(O)CC(F)(F)C[C@@H]1Oc1nc(NC2CCN(S(=O)(=O)CC3CC3)CC2)ncc1C#N]

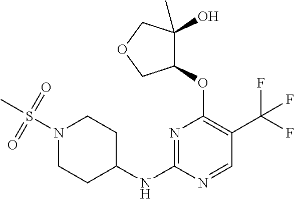 [SMILES: C[C@@]1(O)COC[C@@H]1Oc1nc(NC2CCN(S(C)(=O)=O)CC2)ncc1C(F)(F)F]

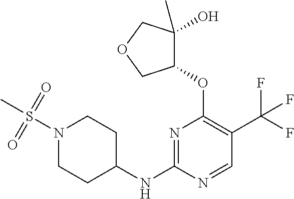 [SMILES: C[C@]1(O)COC[C@H]1Oc1nc(NC2CCN(S(C)(=O)=O)CC2)ncc1C(F)(F)F]

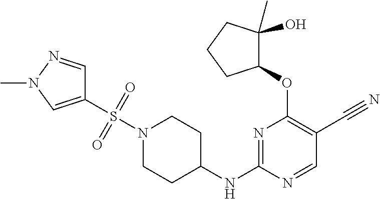 [SMILES: Cn1cc(S(=O)(=O)N2CCC(Nc3ncc(C#N)c(O[C@H]4CCC[C@@]4(C)O)n3)CC2)cn1]

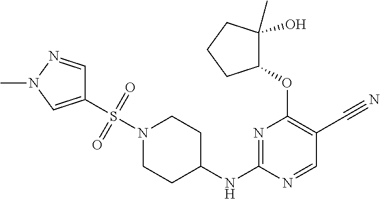 [SMILES: Cn1cc(S(=O)(=O)N2CCC(Nc3ncc(C#N)c(O[C@@H]4CCC[C@]4(C)O)n3)CC2)cn1]

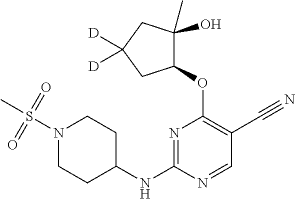 [SMILES: [2H]C1([2H])C[C@H](Oc2nc(NC3CCN(S(C)(=O)=O)CC3)ncc2C#N)[C@](C)(O)C1]

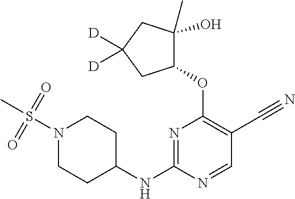 [SMILES: [2H]C1([2H])C[C@@H](Oc2nc(NC3CCN(S(C)(=O)=O)CC3)ncc2C#N)[C@@](C)(O)C1]

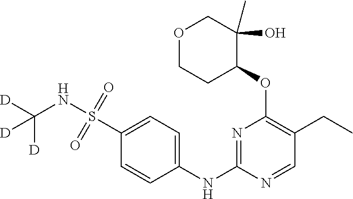 [SMILES: [2H]C([2H])([2H])NS(=O)(=O)c1ccc(Nc2ncc(CC)c(O[C@H]3CCOC[C@@]3(C)O)n2)cc1]

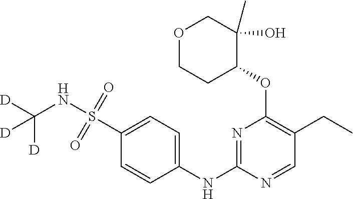 [SMILES: [2H]C([2H])([2H])NS(=O)(=O)c1ccc(Nc2ncc(CC)c(O[C@@H]3CCOC[C@]3(C)O)n2)cc1]

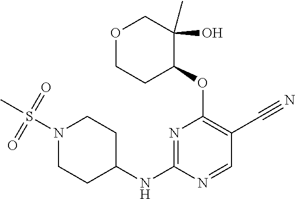 [SMILES: C[C@@]1(O)COCC[C@@H]1Oc1nc(NC2CCN(S(C)(=O)=O)CC2)ncc1C#N]

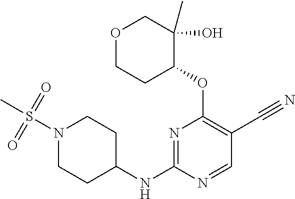 [SMILES: C[C@]1(O)COCC[C@H]1Oc1nc(NC2CCN(S(C)(=O)=O)CC2)ncc1C#N]

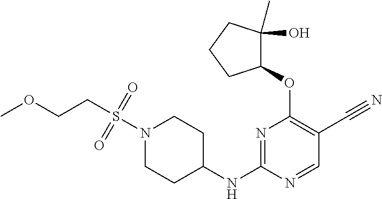 [SMILES: COCCS(=O)(=O)N1CCC(Nc2ncc(C#N)c(O[C@H]3CCC[C@@]3(C)O)n2)CC1]

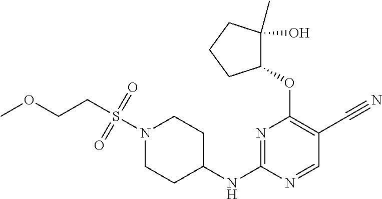 [SMILES: COCCS(=O)(=O)N1CCC(Nc2ncc(C#N)c(O[C@@H]3CCC[C@]3(C)O)n2)CC1]

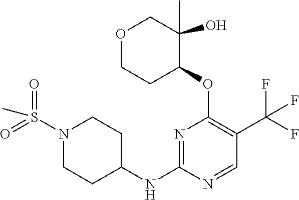 [SMILES: C[C@@]1(O)COCC[C@@H]1Oc1nc(NC2CCN(S(C)(=O)=O)CC2)ncc1C(F)(F)F]

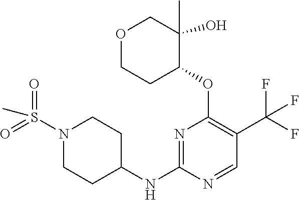 [SMILES: C[C@]1(O)COCC[C@H]1Oc1nc(NC2CCN(S(C)(=O)=O)CC2)ncc1C(F)(F)F]

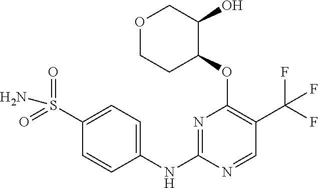 [SMILES: NS(=O)(=O)c1ccc(Nc2ncc(C(F)(F)F)c(O[C@H]3CCOC[C@H]3O)n2)cc1]

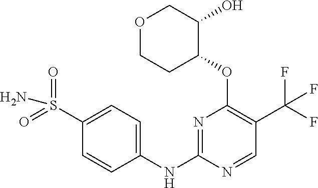 [SMILES: NS(=O)(=O)c1ccc(Nc2ncc(C(F)(F)F)c(O[C@@H]3CCOC[C@@H]3O)n2)cc1]

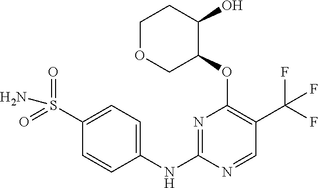 [SMILES: NS(=O)(=O)c1ccc(Nc2ncc(C(F)(F)F)c(O[C@H]3COCC[C@H]3O)n2)cc1]

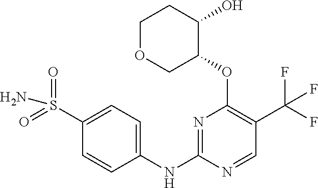 [SMILES: NS(=O)(=O)c1ccc(Nc2ncc(C(F)(F)F)c(O[C@@H]3COCC[C@@H]3O)n2)cc1]

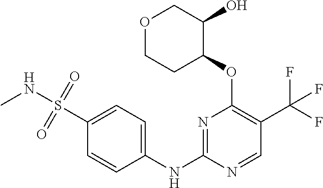 [SMILES: CNS(=O)(=O)c1ccc(Nc2ncc(C(F)(F)F)c(O[C@H]3CCOC[C@H]3O)n2)cc1]

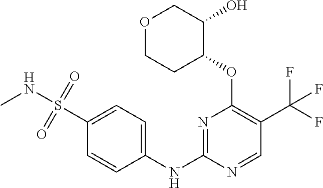 [SMILES: CNS(=O)(=O)c1ccc(Nc2ncc(C(F)(F)F)c(O[C@@H]3CCOC[C@@H]3O)n2)cc1]

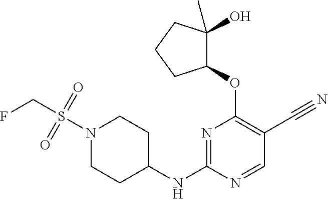 [SMILES: C[C@@]1(O)CCC[C@@H]1Oc1nc(NC2CCN(S(=O)(=O)CF)CC2)ncc1C#N]

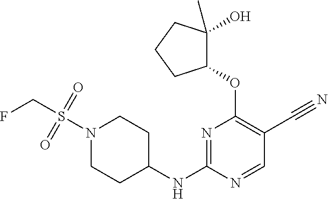 [SMILES: C[C@]1(O)CCC[C@H]1Oc1nc(NC2CCN(S(=O)(=O)CF)CC2)ncc1C#N]

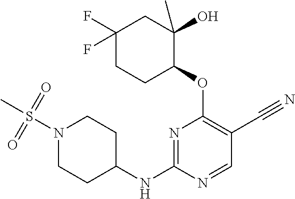 [SMILES: C[C@@]1(O)CC(F)(F)CC[C@@H]1Oc1nc(NC2CCN(S(C)(=O)=O)CC2)ncc1C#N]

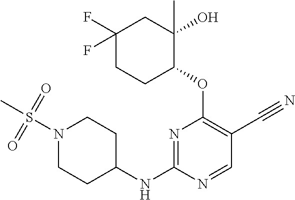 [SMILES: C[C@]1(O)CC(F)(F)CC[C@H]1Oc1nc(NC2CCN(S(C)(=O)=O)CC2)ncc1C#N]

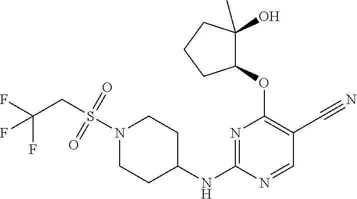 [SMILES: C[C@@]1(O)CCC[C@@H]1Oc1nc(NC2CCN(S(=O)(=O)CC(F)(F)F)CC2)ncc1C#N]

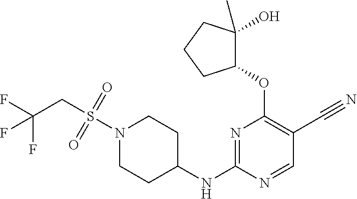 [SMILES: C[C@]1(O)CCC[C@H]1Oc1nc(NC2CCN(S(=O)(=O)CC(F)(F)F)CC2)ncc1C#N]

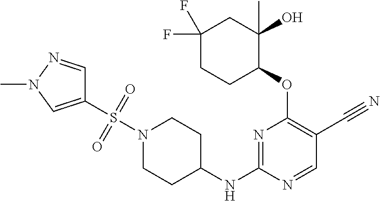 [SMILES: Cn1cc(S(=O)(=O)N2CCC(Nc3ncc(C#N)c(O[C@H]4CCC(F)(F)C[C@@]4(C)O)n3)CC2)cn1]

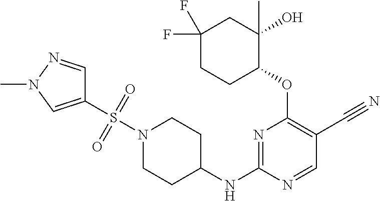 [SMILES: Cn1cc(S(=O)(=O)N2CCC(Nc3ncc(C#N)c(O[C@@H]4CCC(F)(F)C[C@]4(C)O)n3)CC2)cn1]

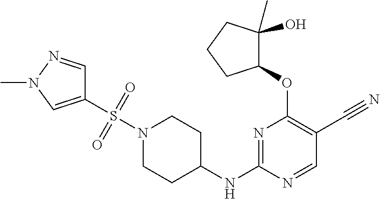 [SMILES: Cn1cc(S(=O)(=O)N2CCC(Nc3ncc(C#N)c(O[C@H]4CCC[C@@]4(C)O)n3)CC2)cn1]

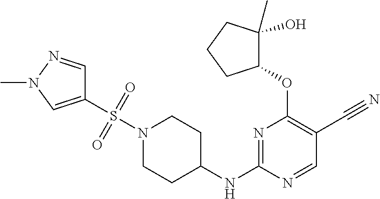 [SMILES: Cn1cc(S(=O)(=O)N2CCC(Nc3ncc(C#N)c(O[C@@H]4CCC[C@]4(C)O)n3)CC2)cn1]

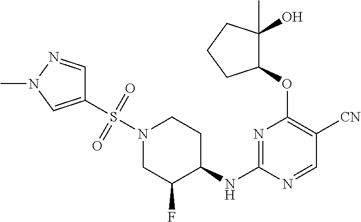 [SMILES: Cn1cc(S(=O)(=O)N2CC[C@@H](Nc3ncc(C#N)c(O[C@H]4CCC[C@@]4(C)O)n3)[C@@H](F)C2)cn1]

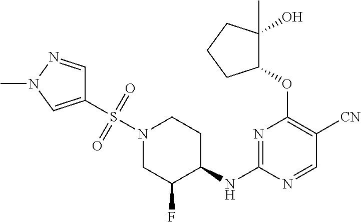 [SMILES: Cn1cc(S(=O)(=O)N2CC[C@@H](Nc3ncc(C#N)c(O[C@@H]4CCC[C@]4(C)O)n3)[C@@H](F)C2)cn1]

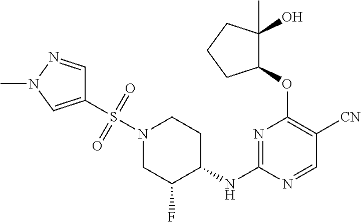 [SMILES: Cn1cc(S(=O)(=O)N2CC[C@H](Nc3ncc(C#N)c(O[C@H]4CCC[C@@]4(C)O)n3)[C@H](F)C2)cn1]

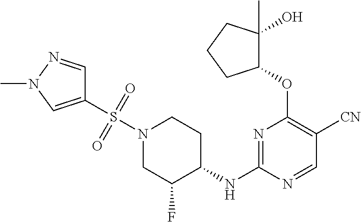 [SMILES: Cn1cc(S(=O)(=O)N2CC[C@H](Nc3ncc(C#N)c(O[C@@H]4CCC[C@]4(C)O)n3)[C@H](F)C2)cn1]

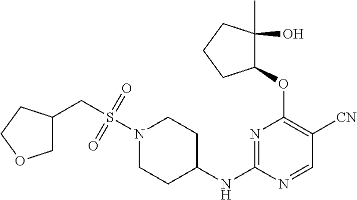 [SMILES: C[C@@]1(O)CCC[C@@H]1Oc1nc(NC2CCN(S(=O)(=O)CC3CCOC3)CC2)ncc1C#N]

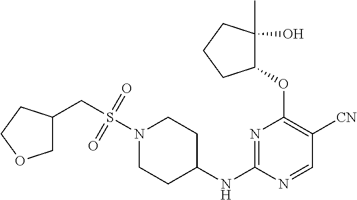 [SMILES: C[C@]1(O)CCC[C@H]1Oc1nc(NC2CCN(S(=O)(=O)CC3CCOC3)CC2)ncc1C#N]

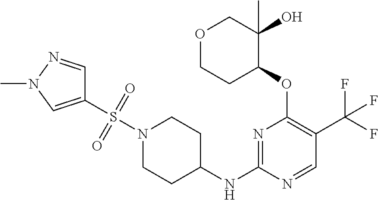 [SMILES: Cn1cc(S(=O)(=O)N2CCC(Nc3ncc(C(F)(F)F)c(O[C@H]4CCOC[C@@]4(C)O)n3)CC2)cn1]

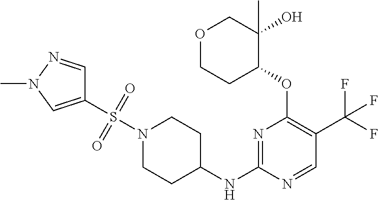 [SMILES: Cn1cc(S(=O)(=O)N2CCC(Nc3ncc(C(F)(F)F)c(O[C@@H]4CCOC[C@]4(C)O)n3)CC2)cn1]

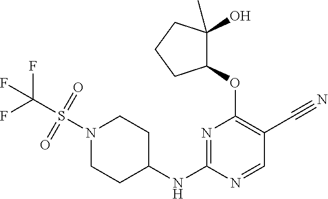 [SMILES: C[C@@]1(O)CCC[C@@H]1Oc1nc(NC2CCN(S(=O)(=O)C(F)(F)F)CC2)ncc1C#N]

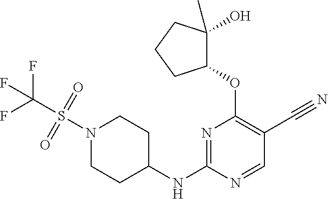 [SMILES: C[C@]1(O)CCC[C@H]1Oc1nc(NC2CCN(S(=O)(=O)C(F)(F)F)CC2)ncc1C#N]

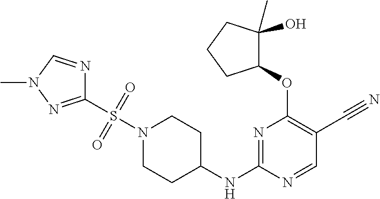 [SMILES: Cn1cnc(S(=O)(=O)N2CCC(Nc3ncc(C#N)c(O[C@H]4CCC[C@@]4(C)O)n3)CC2)n1]

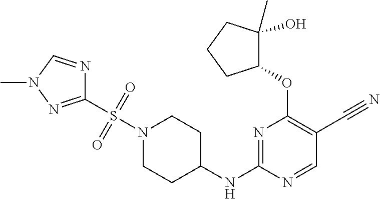 [SMILES: Cn1cnc(S(=O)(=O)N2CCC(Nc3ncc(C#N)c(O[C@@H]4CCC[C@]4(C)O)n3)CC2)n1]

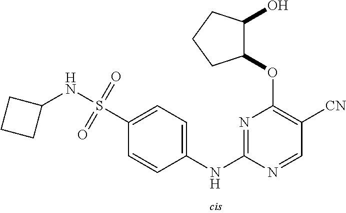

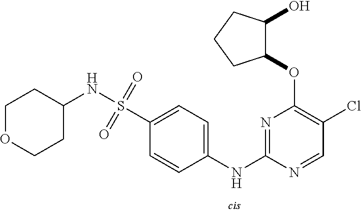

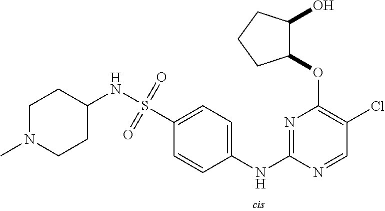

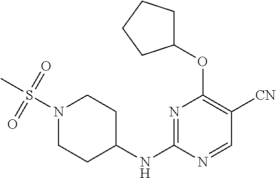 [SMILES: CS(=O)(=O)N1CCC(Nc2ncc(C#N)c(OC3CCCC3)n2)CC1]

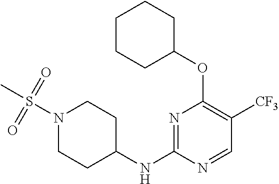 [SMILES: CS(=O)(=O)N1CCC(Nc2ncc(C(F)(F)F)c(OC3CCCCC3)n2)CC1]

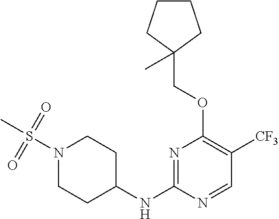 [SMILES: CC1(COc2nc(NC3CCN(S(C)(=O)=O)CC3)ncc2C(F)(F)F)CCCC1]

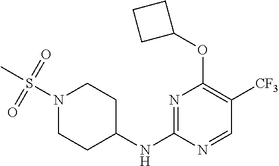 [SMILES: CS(=O)(=O)N1CCC(Nc2ncc(C(F)(F)F)c(OC3CCC3)n2)CC1]

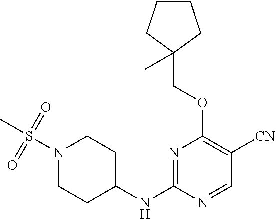 [SMILES: CC1(COc2nc(NC3CCN(S(C)(=O)=O)CC3)ncc2C#N)CCCC1]

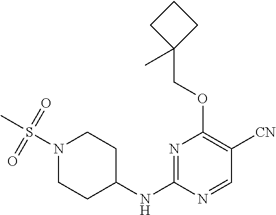 [SMILES: CC1(COc2nc(NC3CCN(S(C)(=O)=O)CC3)ncc2C#N)CCC1]

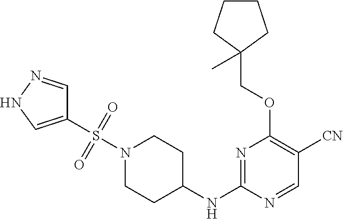 [SMILES: CC1(COc2nc(NC3CCN(S(=O)(=O)c4cn[nH]c4)CC3)ncc2C#N)CCCC1]

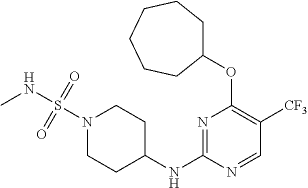 [SMILES: CNS(=O)(=O)N1CCC(Nc2ncc(C(F)(F)F)c(OC3CCCCCC3)n2)CC1]

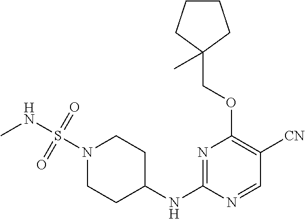 [SMILES: CNS(=O)(=O)N1CCC(Nc2ncc(C#N)c(OCC3(C)CCCC3)n2)CC1]

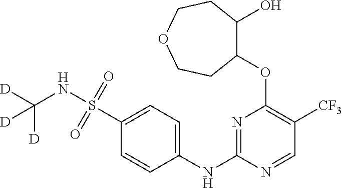 [SMILES: [2H]C([2H])([2H])NS(=O)(=O)c1ccc(Nc2ncc(C(F)(F)F)c(OC3CCOCCC3O)n2)cc1]

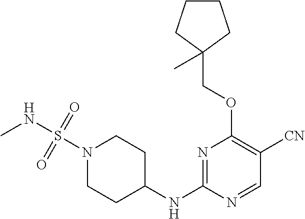 [SMILES: CNS(=O)(=O)N1CCC(Nc2ncc(C#N)c(OCC3(C)CCCC3)n2)CC1]

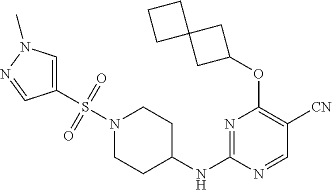 [SMILES: Cn1cc(S(=O)(=O)N2CCC(Nc3ncc(C#N)c(OC4CC5(CCC5)C4)n3)CC2)cn1]

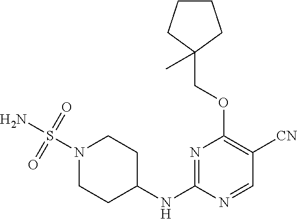 [SMILES: CC1(COc2nc(NC3CCN(S(N)(=O)=O)CC3)ncc2C#N)CCCC1]

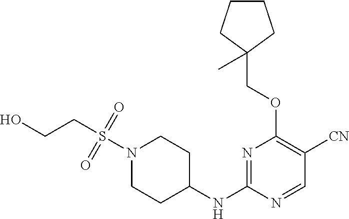 [SMILES: CC1(COc2nc(NC3CCN(S(=O)(=O)CCO)CC3)ncc2C#N)CCCC1]

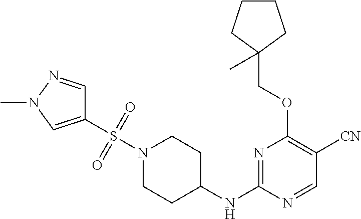 [SMILES: Cn1cc(S(=O)(=O)N2CCC(Nc3ncc(C#N)c(OCC4(C)CCCC4)n3)CC2)cn1]

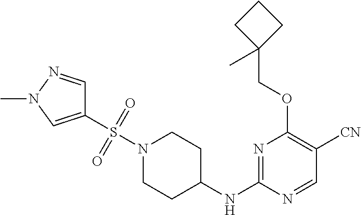 [SMILES: Cn1cc(S(=O)(=O)N2CCC(Nc3ncc(C#N)c(OCC4(C)CCC4)n3)CC2)cn1]

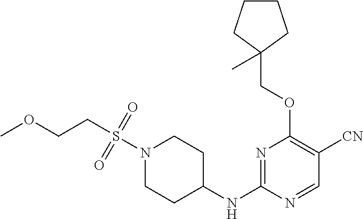 [SMILES: COCCS(=O)(=O)N1CCC(Nc2ncc(C#N)c(OCC3(C)CCCC3)n2)CC1]

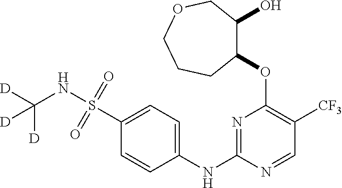

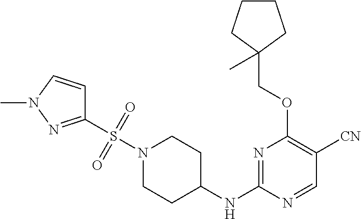 [SMILES: Cn1ccc(S(=O)(=O)N2CCC(Nc3ncc(C#N)c(OCC4(C)CCCC4)n3)CC2)n1]

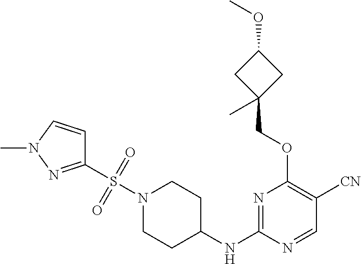 [SMILES: CO[C@H]1C[C@](C)(COc2nc(NC3CCN(S(=O)(=O)c4ccn(C)n4)CC3)ncc2C#N)C1]

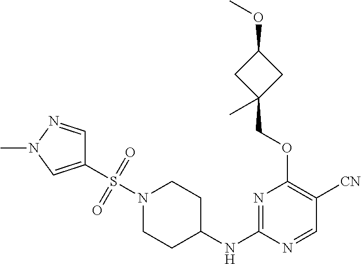 [SMILES: CO[C@H]1C[C@@](C)(COc2nc(NC3CCN(S(=O)(=O)c4cnn(C)c4)CC3)ncc2C#N)C1]

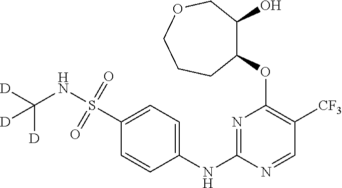 [SMILES: [2H]C([2H])([2H])NS(=O)(=O)c1ccc(Nc2ncc(C(F)(F)F)c(O[C@H]3CCCOC[C@H]3O)n2)cc1]

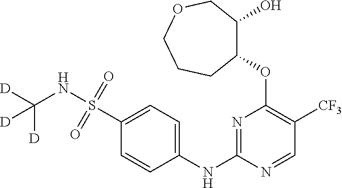 [SMILES: [2H]C([2H])([2H])NS(=O)(=O)c1ccc(Nc2ncc(C(F)(F)F)c(O[C@@H]3CCCOC[C@@H]3O)n2)cc1]

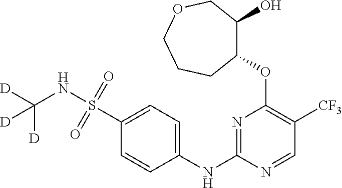 [SMILES: [2H]C([2H])([2H])NS(=O)(=O)c1ccc(Nc2ncc(C(F)(F)F)c(O[C@@H]3CCCOC[C@H]3O)n2)cc1]

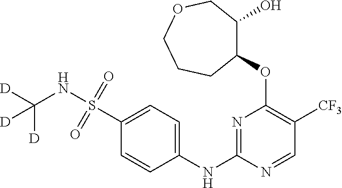 [SMILES: [2H]C([2H])([2H])NS(=O)(=O)c1ccc(Nc2ncc(C(F)(F)F)c(O[C@H]3CCCOC[C@@H]3O)n2)cc1]

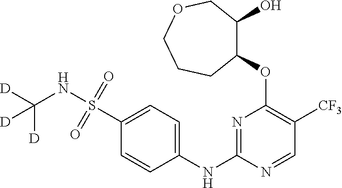 [SMILES: [2H]C([2H])([2H])NS(=O)(=O)c1ccc(Nc2ncc(C(F)(F)F)c(O[C@H]3CCCOC[C@H]3O)n2)cc1]

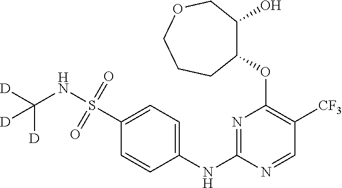 [SMILES: [2H]C([2H])([2H])NS(=O)(=O)c1ccc(Nc2ncc(C(F)(F)F)c(O[C@@H]3CCCOC[C@@H]3O)n2)cc1]

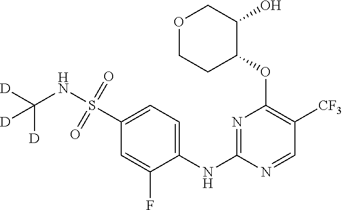 [SMILES: [2H]C([2H])([2H])NS(=O)(=O)c1ccc(Nc2ncc(C(F)(F)F)c(O[C@@H]3CCOC[C@@H]3O)n2)c(F)c1]

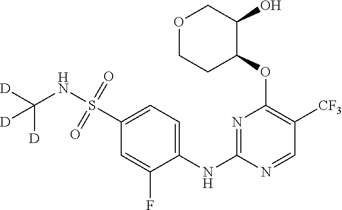 [SMILES: [2H]C([2H])([2H])NS(=O)(=O)c1ccc(Nc2ncc(C(F)(F)F)c(O[C@H]3CCOC[C@H]3O)n2)c(F)c1]

Compounds of Table 1 can exist in various stereoisomeric forms, such as individual isomer, an individual enantiomer and/or diastereomer, as applicable, or a mixture of stereoisomers, including racemic mixtures and mixtures enriched in one or more stereoisomers. In some embodiments, when applicable, a compound shown Table 1 can exist as an isolated individual enantiomer substantially free (e.g., with less than 20%, less than 10%, less than 5%, less than 1%, by weight, by HPLC or SFC area, or both, or with a non-detectable amount) of the other enantiomer. In some embodiments, when applicable, a compound shown Table 1 can also exist as a mixture of stereoisomers in any ratio, such as a racemic mixture.

In some embodiments, the present disclosure provides a compound of 4-((4-(((3S,4R)-3-hydroxytetrahydro-2H-pyran-4-yl)oxy)-5-(trifluoromethyl)pyrimidin-2-yl)amino)-N-(methyl-d3)benzenesulfonamide.

In some embodiments, the present disclosure provides a compound of 4-((4-(((3R,4R)-3-hydroxytetrahydro-2H-pyran-4-yl)oxy)-5-(trifluoromethyl)pyrimidin-2-yl)amino)-N-(methyl-d3)benzenesulfonamide.

In some embodiments, the present disclosure provides a compound of 4-((4-(((3S,4R)-3-hydroxytetrahydro-2H-pyran-4-yl)oxy)-5-(trifluoromethyl)pyrimidin-2-yl)amino)-N-methylbenzenesulfonamide.

In some embodiments, the present disclosure provides a compound of N-ethyl-4-((4-(((3S,4R)-3-hydroxytetrahydro-2H-pyran-4-yl)oxy)-5-(trifluoromethyl)pyrimidin-2-yl)amino)benzenesulfonamide.

In some embodiments, the present disclosure provides a compound of 4-((4-(((3S,4R)-3-hydroxytetrahydro-2H-pyran-4-yl)oxy)-5-(trifluoromethyl)pyrimidin-2-yl)amino)benzenesulfonamide.

The compounds of the present disclosure can be readily synthesized by those skilled in the art in view of the present disclosure. Exemplified synthesis are also shown in the Examples section. Novel synthetic intermediates described herein are also one aspect of this disclosure. For example, in some embodiments, the present disclosure provides a compound selected from: 4-((4-(((3S,4R)-3-((4-methoxybenzyl)oxy)tetrahydro-2H-pyran-4-yl)oxy)-5-(trifluoromethyl)pyrimidin-2-yl)amino)benzenesulfonamide; 4-((4-(((3S,4R)-3-((4-methoxybenzyl)oxy)tetrahydro-2H-pyran-4-yl)oxy)-5-(trifluoromethyl)pyrimidin-2-yl)amino)-N-methylbenzenesulfonamide; N-ethyl-4-((4-(((3S,4R)-3-((4-methoxybenzyl)oxy)tetrahydro-2H-pyran-4-yl)oxy)-5-(trifluoromethyl)pyrimidin-2-yl)amino)benzenesulfonamide; 4-((4-(((3S,4R)-3-((4-methoxybenzyl)oxy)tetrahydro-2H-pyran-4-yl)oxy)-5-(trifluoromethyl)pyrimidin-2-yl)amino)-N-(methyl-d3)benzenesulfonamide; 4-((4-(((3R,4R)-3-((4-methoxybenzyl)oxy)tetrahydro-2H-pyran-4-yl)oxy)-5-(trifluoromethyl)pyrimidin-2-yl)amino)-N-(methyl-d3)benzenesulfonamide; 4-((4-(((3S,4S)-3-((4-methoxybenzyl)oxy)tetrahydro-2H-pyran-4-yl)oxy)-5-(trifluoromethyl)pyrimidin-2-yl)amino)-N-(methyl-d3)benzenesulfonamide; and 4-((4-(((3R,4S)-3-((4-methoxybenzyl)oxy)tetrahydro-2H-pyran-4-yl)oxy)-5-(trifluoromethyl)pyrimidin-2-yl)amino)-N-(methyl-d3)benzenesulfonamide.

### Pharmaceutical Compositions

Certain embodiments are directed to a pharmaceutical composition comprising one or more compounds of the present disclosure.

The pharmaceutical composition can optionally contain a pharmaceutically acceptable excipient. In some embodiments, the pharmaceutical composition comprises a compound of the present disclosure (e.g., any of Examples E1-E7, or any of the specific compounds disclosed in Table 1 herein, or a pharmaceutically acceptable salt thereof) and a pharmaceutically acceptable excipient. Pharmaceutically acceptable excipients are known in the art. Non-limiting suitable excipients include, for example, encapsulating materials or additives such as antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises any of Examples E1-E7, or any of the specific compounds disclosed in Table 1 herein, or a pharmaceutically acceptable salt thereof, e.g., in a therapeutically effective amount. In any of the embodiments described herein, the pharmaceutical composition can comprise a therapeutically effective amount (e.g., for treating breast cancer or ovarian cancer) of a compound selected from any of Examples E1-E7, or any of the specific compounds disclosed in Table 1 herein, or a pharmaceutically acceptable salt thereof. In some preferred embodiments, the pharmaceutical composition can comprise a compound selected from the compounds according to Examples E1-E7 or those in Table 1 herein that have a CDK2/CyclinE1 IC50 level of less than 100 nM, more preferably, less than 10 nM, when tested according to Biological Example 1 as described in WO2022/111621.

The pharmaceutical composition herein can be formulated for delivery via any of the known routes of delivery, which include but not limited to administering orally, nasally, transdermally, pulmonary, inhalationally, buccally, sublingually, intraperintoneally, subcutaneously, intramuscularly, intravenously, rectally, intrapleurally, intrathecally or parenterally.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion. Excipients for the preparation of parenteral formulations are known in the art. Non-limiting suitable excipients include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof.

Compounds of the present disclosure can be used alone, in combination with each other, or in combination with one or more additional therapeutic agents, e.g., in combination with an additional anticancer therapeutic agent, such as mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, immuno-oncology agents, and the like. In some embodiments, one or more compounds of the present disclosure can be used in combination with one or more targeted agents, such as inhibitors of PI3 kinase, mTOR, PARP, IDO, TDO, ALK, ROS, MEK, VEGF, FLT3, AXL, ROR2, EGFR, FGFR, Src/Abl, RTK/Ras, Myc, Raf, PDGF, AKT, c-Kit, erbB, CDK4/CDK6, CDK5, CDK7, CDK9, SMO, CXCR4, HER2, GLS1, EZH2 or Hsp90, or immunomodulatory agents, such as PD-1 or PD-L1 antagonists, OX40 agonists or 4-1BB agonists. In some embodiments, one or more compounds of the present disclosure can be used in combination with a standard of care agent, such as tamoxifen, docetaxel, paclitaxel, cisplatin, capecitabine, gemcitabine, vinorelbine, exemestane, letrozole, fulvestrant, anastrozole or trastuzumab. Suitable additional anticancer therapeutic agent include any of those known in the art, such as those approved for the appropriate cancer by a regulatory agency such as the U.S. Food and Drug Administration. Some examples of suitable additional anticancer therapeutic agents also include those described in WO2020/157652, US2018/0044344, WO2008/122767, etc., the content of each of which is herein incorporated by reference in its entireties.

When used in combination with one or more additional therapeutic agents, compounds of the present disclosure or pharmaceutical compositions herein can be administered to the subject either concurrently or sequentially in any order with such additional therapeutic agents. In some embodiments, the pharmaceutical composition can comprise one or more compounds of the present disclosure and the one or more additional therapeutic agents in a single composition. In some embodiments, the pharmaceutical composition comprising one or more compounds of the present disclosure can be included in a kit which also comprises a separate pharmaceutical composition comprising the one or more additional therapeutic agents.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure. In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disease or disorder as described herein, such as breast cancer or ovarian cancer, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Method of Treatment/Use

Compounds of the present disclosure have various utilities. For example, compounds of the present disclosure can be used as therapeutic active substances for the treatment and/or prophylaxis of a CDK2-mediated disease or disorder. Accordingly, some embodiments of the present disclosure are also directed to methods of using one or more compounds of the present disclosure or pharmaceutical compositions herein for treating or preventing a CDK2-mediated disease or disorder in a subject in need thereof, such as for treating cancer in a subject in need thereof.

In some embodiments, the present disclosure provides a method of inhibiting abnormal cell growth in a subject in need thereof, comprising administering to the subject a therapeutically effective amount of a compound of the present disclosure or a pharmaceutical composition described herein. In some embodiments, the abnormal cell growth is cancer characterized by amplification or overexpression of cyclin E1 (CCNE1) and/or cyclin E2 (CCNE2). In some embodiments, the subject is identified as having a cancer characterized by amplification or overexpression of CCNE1 and/or CCNE2.

In some embodiments, the present disclosure also provides a method of inhibiting CDK activity in a subject or biological sample. In some embodiments, the present disclosure provides a method of inhibiting CDK2 activity in a subject or biological sample, which comprises contacting the subject or biological sample with an effective amount of the compound of the present disclosure (e.g., any of Examples E1-E7, or any of the specific compounds disclosed in Table 1 herein, or a pharmaceutically acceptable salt thereof) or a pharmaceutical composition described herein.

In some embodiments, the present disclosure provides a method of treating or preventing a CDK mediated, in particular CDK2-mediated disease or disorder in a subject in need thereof. In some embodiments, the method comprises administering to the subject an effective amount of a compound of the present disclosure (e.g., any of Examples E1-E7, or any of the specific compounds disclosed in Table 1 herein, or a pharmaceutically acceptable salt thereof) or an effective amount of a pharmaceutical composition described herein. In some embodiments, the CDK2-mediated disease or disorder is cancer. In some embodiments, the cancer is characterized by amplification or overexpression of CCNE1 and/or CCNE2

In some embodiments, the present disclosure also provides a method of treating or preventing cancer in a subject in need thereof, which comprises administering to the subject an effective amount of a compound of the present disclosure (e.g., any of Examples E1-E7, or any of the specific compounds disclosed in Table 1 herein, or a pharmaceutically acceptable salt thereof) or an effective amount of a pharmaceutical composition described herein. In some embodiments, the cancer is characterized by amplification or overexpression of CCNE1 and/or CCNE2. In some embodiments, the subject is identified as having a cancer characterized by amplification or overexpression of CCNE1 and/or CCNE2. In some embodiments, the cancer is selected from breast cancer, ovarian cancer, bladder cancer, uterine cancer, prostate cancer, lung cancer (including NSCLC, SCLC, squamous cell carcinoma or adenocarcinoma), esophageal cancer, head and neck cancer, colorectal cancer, kidney cancer (including RCC), liver cancer (including HCC), pancreatic cancer, stomach (i.e., gastric) cancer, thyroid cancer, and combinations thereof. In some embodiments of the methods herein, the cancer is breast cancer, ovarian cancer, bladder cancer, uterine cancer, prostate cancer, lung cancer, esophageal cancer, liver cancer, pancreatic cancer and/or stomach cancer.

In some embodiments of the methods herein, the cancer is breast cancer, such as ER-positive/HR-positive, HER2-negative breast cancer; ER-positive/HR-positive, HER2-positive breast cancer; triple negative breast cancer (TNBC); or inflammatory breast cancer. In some embodiments, the breast cancer can be endocrine resistant breast cancer, trastuzumab resistant breast cancer, or breast cancer demonstrating primary or acquired resistance to CDK4/CDK6 inhibition. In some embodiments, the breast cancer can be advanced or metastatic breast cancer. In some embodiments, the breast cancer described herein is characterized by amplification or overexpression of CCNE1 and/or CCNE2.

In some embodiments of the methods herein, the cancer is ovarian cancer. In some embodiments, the ovarian cancer is characterized by amplification or overexpression of CCNE1 and/or CCNE2.

In some embodiments of the methods herein, the cancer is blood cancer such as leukemia. In some embodiments of the methods herein, the cancer is chronic lymphocytic leukemia, such as relapsed or refractory Chronic Lymphocytic Leukemia (CLL).

In some embodiments of the methods herein, the cancer is acute myeloid leukemia. In some embodiments of the methods herein, the cancer is relapsed or refractory Acute Myeloid Leukemia or Myelodysplastic Syndromes.

In any of the embodiments described herein, unless otherwise specified or contradictory, the cancer herein can be characterized by amplification or overexpression of CCNE1 and/or CCNE2.

In some embodiments, the present disclosure also provides a method of treating breast cancer in a subject in need thereof, which comprises administering to the subject a therapeutically effective amount of a compound of the present disclosure (e.g., any of Examples E1-E7, or any of the specific compounds disclosed in Table 1 herein, or a pharmaceutically acceptable salt thereof) or an effective amount of a pharmaceutical composition described herein. In some embodiments, the breast cancer is selected from ER-positive/HR-positive, HER2-negative breast cancer; ER-positive/HR-positive, HER2-positive breast cancer; triple negative breast cancer (TNBC); and inflammatory breast cancer. In some embodiments, the breast cancer is selected from endocrine resistant breast cancer, trastuzumab resistant breast cancer, or breast cancer demonstrating primary or acquired resistance to CDK4/CDK6 inhibition. In some embodiments, the breast cancer is advanced or metastatic breast cancer. In some embodiments, the breast cancer is characterized by amplification or overexpression of CCNE1 and/or CCNE2.

In some embodiments, the present disclosure also provides a method of treating ovarian cancer in a subject in need thereof, which comprises administering to the subject a therapeutically effective amount of a compound of the present disclosure (e.g., any of Examples E1-E7, or any of the specific compounds disclosed in Table 1 herein, or a pharmaceutically acceptable salt thereof) or an effective amount of a pharmaceutical composition described herein. In some embodiments, the ovarian cancer is characterized by amplification or overexpression of CCNE1 and/or CCNE2.

In some embodiments, the present disclosure also provides a method of treating leukemia in a subject in need thereof, which comprises administering to the subject a therapeutically effective amount of a compound of the present disclosure (e.g., any of Examples E1-E7, or any of the specific compounds disclosed in Table 1 herein, or a pharmaceutically acceptable salt thereof) or an effective amount of a pharmaceutical composition described herein. In some embodiments, the leukemia is characterized by amplification or overexpression of CCNE1 and/or CCNE2.

In some embodiments, the present disclosure also provides a method of treating chronic lymphocytic leukemia, such as relapsed or refractory Chronic Lymphocytic Leukemia (CLL), in a subject in need thereof, which comprises administering to the subject a therapeutically effective amount of a compound of the present disclosure (e.g., any of Examples E1-E7, or any of the specific compounds disclosed in Table 1 herein, or a pharmaceutically acceptable salt thereof) or an effective amount of a pharmaceutical composition described herein.

In some embodiments, the present disclosure also provides a method of treating acute myeloid leukemia, such as relapsed or refractory Acute Myeloid Leukemia, in a subject in need thereof, which comprises administering to the subject a therapeutically effective amount of a compound of the present disclosure (e.g., any of Examples E1-E7, or any of the specific compounds disclosed in Table 1 herein, or a pharmaceutically acceptable salt thereof) or an effective amount of a pharmaceutical composition described herein.

In some embodiments, the present disclosure also provides a method of treating Myelodysplastic Syndromes in a subject in need thereof, which comprises administering to the subject a therapeutically effective amount of a compound of the present disclosure (e.g., any of Examples E1-E7, or any of the specific compounds disclosed in Table 1 herein, or a pharmaceutically acceptable salt thereof) or an effective amount of a pharmaceutical composition described herein.

In some preferred embodiments, the compound of the present disclosure for the methods herein has a CDK2/CyclinE1 IC50 of less than 100 nM, more preferably, less than 10 nM, measured/calculated according to the Biological Example 1 as described in WO2022/111621.

The administering in the methods herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the administering is orally. In some embodiments, the administering is a parenteral injection, such as an intraveneous injection.

Compounds of the present disclosure can be used as a monotherapy or in a combination therapy. In some embodiments according to the methods described herein, one or more compounds of the present disclosure can be administered as the only active ingredient(s). In some embodiments according to the methods described herein, one or more compounds of the present disclosure can also be co-administered with an additional therapeutic agent, either concurrently or sequentially in any order, to the subject in need thereof. The additional therapeutic agent can typically be an additional anticancer therapeutic agent, such as mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, immuno-oncology agents, and the like. In some embodiments, the additional anticancer agent is an endocrine agent, such as an aromatase inhibitor, a SERD or a SERM. In some embodiments, one or more compounds of the present disclosure can be administered in combination with one or more targeted agents, such as inhibitors of PI3 kinase, mTOR, PARP, IDO, TDO, ALK, ROS, MEK, VEGF, FLT3, AXL, ROR2, EGFR, FGFR, Src/Abl, RTK/Ras, Myc, Raf, PDGF, AKT, c-Kit, erbB, CDK4/CDK6, CDK5, CDK7, CDK9, SMO, CXCR4, HER2, GLS1, EZH2 or Hsp90, or immunomodulatory agents, such as PD-1 or PD-L1 antagonists, OX40 agonists or 4-1BB agonists. In some embodiments, one or more compounds of the present disclosure can be administered administered in combination with a standard of care agent, such as tamoxifen, docetaxel, paclitaxel, cisplatin, capecitabine, gemcitabine, vinorelbine, exemestane, letrozole, fulvestrant, anastrozole or trastuzumab. Suitable additional anticancer therapeutic agent include any of those known in the art, such as those approved for the appropriate cancer by a regulatory agency such as the U.S. Food and Drug Administration. Some examples of suitable additional anticancer therapeutic agents also include those described in WO2020/157652, US2018/0044344, WO2008/122767, etc., the contents of each of which is incorporated by reference herein in their entirety.

Dosing regimen including doses for the methods described herein can vary and be adjusted, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

It is also meant to be understood that a specific embodiment of a variable moiety herein can be the same or different as another specific embodiment having the same identifier.

The symbol, [symbol] whether utilized as a bond or displayed perpendicular to (or otherwise crossing) a bond, indicates the point at which the displayed moiety is attached to the remainder of the molecule. It should be noted that the immediately connected group or groups maybe shown beyond the symbol, [symbol] , to indicate connectivity, as would be understood by those skilled in the art.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC), chiral supercritical fluid chromatograph (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures. When a stereochemistry is specifically drawn, unless otherwise contradictory from context, it should be understood that with respect to that particular chiral center or axial chirality, the compound can exist predominantly as the as-drawn stereoisomer, such as with less than 20%, less than 10%, less than 5%, less than 1%, by weight, by HPLC or SFC area, or both, or with a non-detectable amount of the other stereoisomer(s). The presence and/or amounts of stereoisomers can be determined by those skilled in the art in view of the present disclosure, including through the use of a chiral HPLC or chiral SFC. As understood by those skilled in the art, when a “*” is shown in the chemical structures herein, unless otherwise contradictory from context, it is to designate that the corresponding chiral center is enantiomerically pure or enriched in either of the configurations or is enantiomerically pure or enriched in the as-dawn configuration, such as with less than 20%, less than 10%, less than 5%, less than 1%, by weight, by HPLC or SFC area, or both, or with a non-detectable amount of the other stereoisomer(s). Also, when no stereochemistry is specifically drawn, and no “*” is used in the chemical structures, unless otherwise contradictory from context, it should be understood that such structures include the corresponding compound in any stereoisomeric forms, including individual isomers substantially free of other isomers and mixtures of various isomers including racemic mixtures.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the term “compound(s) of the present disclosure” refers to any of the compounds described herein according to any of Examples E1-E7, or any of the specific compounds disclosed in Table 1 herein, isotopically labeled compound(s) thereof (such as a deuterated analog wherein one or more of the hydrogen atoms is/are substituted with a deuterium atom with an abundance above its natural abundance, e.g., a CD₃ analog when the compound has a CH₃ group), possible regioisomers, possible geometric isomers, possible stereoisomers thereof (including diastereoisomers, enantiomers, and racemic mixtures), tautomers thereof, conformational isomers thereof, pharmaceutically acceptable esters thereof, and/or possible pharmaceutically acceptable salts thereof (e.g., acid addition salt such as HCl salt or base addition salt such as Na salt). To be clear, compounds of Examples E1-E7 refer to the compounds in the Examples section labeled with E followed by an integer, such as E1, E2, etc. up to E7. Hydrates and solvates of the compounds of the present disclosure are considered compositions of the present disclosure, wherein the compound(s) is in association with water or solvent, respectively.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art.

The term “tautomers” or “tautomeric” refers to two or more interconvertible compounds resulting from tautomerization. The exact ratio of the tautomers depends on several factors, including for example temperature, solvent, and pH. Tautomerizations are known to those skilled in the art. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

The term “effective amount” refers to that amount of a compound or combination of compounds as described herein that is sufficient to effect the intended application including, but not limited to, prophylaxis or treatment of diseases. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells and/or tissues. The specific dose will vary depending on the particular compounds chosen, the dosing regimen to be followed, whether the compound is administered in combination with other compounds, timing of administration, the tissue to which it is administered, and the physical delivery system in which the compound is carried.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### EXAMPLES

The various starting materials, intermediates, and compounds of embodiments herein can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. The abbreviations used in the Examples section should be understood as having their ordinary meanings in the art unless specifically indicated otherwise or obviously contrary from context. The examples are illustrative only and do not limit the claimed invention in any way.

Synthetic procedures for representative compounds of the present disclosure were described in details in PCT/CN2021/133429, published as WO2022/111621 on Jun. 2, 2022, the entire contents of which are incorporated herein by reference. Additional synthetic procedures using chiral starting material/intermediate are described herein below.

### Synthesis of (3S,4R)-3-((4-methoxybenzyl)oxy)tetrahydro-2H-pyran-4-ol

### Intermediate I

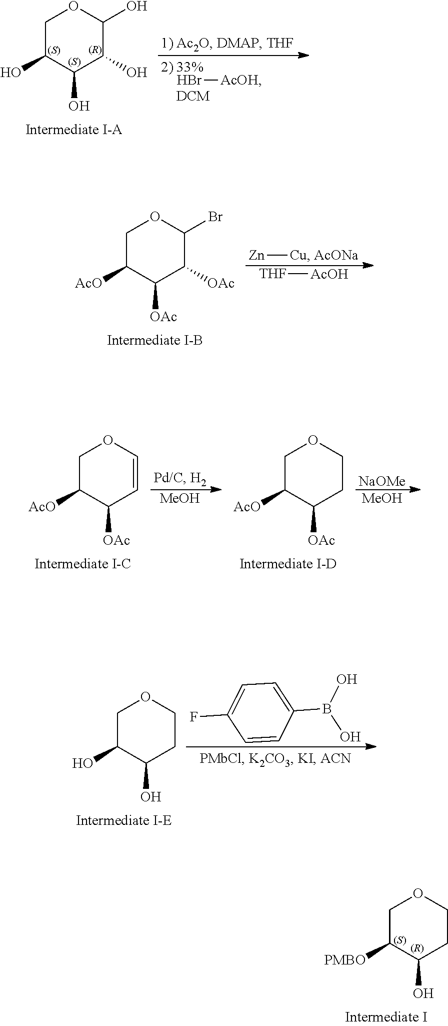

To a mixture of (3R,4S,5S)-tetrahydro-2H-pyran-2,3,4,5-tetraol (Intermediate I-A, 100 g, 0.67 mol) and 4-dimethylaminopyridine (8.18 g) in tetrahydrofuran (400 mL) was added acetic anhydride (328.5 g) dropwise at 10-20° C. under nitrogen atmosphere, and the reaction mixture was stirred for 20 hrs at 20-30° C. The reaction mixture was concentrated under reduced pressure, and then diluted with ethyl acetate (800 mL) and ice water (800 mL). The organic phase was washed with sat. sodium bicarbonate aqueous solution (5%, 800 mL) and brine (15%, 800 mL), and then concentrated under reduced pressure. The residue was dissolved with dichloromethane (800 mL) and cooled to −5-5° C. To the above solution was added hydrobromic acid (33% in acetic acid, 328 g) and it was stirred at -5-5° C. for 18 hrs. The reaction mixture was diluted with ice water (800 mL) below 10° C., and the organic phase was washed with brine (15%, 800 mL) and concentrated under reduced pressure to afford (3R,4S,5S)-2-bromotetrahydro-2H-pyran-3,4,5-triyl triacetate (Intermediate I-B, 124 g, 55%) as a pale yellow solid.

To a mixture of copper acetate (53.6 g, 0.29 mol) in acetic acid (200 mL) and tetrahydrofuran (200 mL) was added zinc dust (115 g, 1.77 mol) in portions at 40-50° C. under nitrogen atmosphere. The mixture was then cooled to 0-5° C., and to it were added tetrahydrofuran (1.30 L), sodium acetate (24.2 g, 0.29 mol) and (3R,4S,5S)-2-bromotetrahydro-2H-pyran-3,4,5-triyl triacetate (Intermediate I-B, 100 g, 0.29 mol). The resulting mixture was stirred at 15-20° C. for 16 hrs under nitrogen atmosphere. The reaction mixture was filtered, and the filtrate was washed with sat. sodium carbonate aqueous solution (2.0 L) and water (500 mL). The organic phase was concentrated under reduced pressure to afford (3S,4R)-3,4-dihydro-2H-pyran-3,4-diyl diacetate (Intermediate I-C, 48.4 g, 82%) as a colorless oil.

To a solution of (3S,4R)-3,4-dihydro-2H-pyran-3,4-diyl diacetate (Intermediate I-C, 500 g, 2.50 mol) in methyl alcohol (3.5 L) was add palladium (50 g, 5% on carbon) under nitrogen atmosphere, and then the mixture was stirred at 25° C. for 26 hrs under hydrogen atmosphere (1 atm). The reaction mixture was filtered and the filter cake was washed with methyl alcohol (500 mL). The filtrates were concentrated under reduced pressure to afford (3S,4R)-tetrahydro-2H-pyran-3,4-diyl diacetate (Intermediate I-D, 480 g, 95%) as a colorless oil.

To a solution of (3S,4R)-tetrahydro-2H-pyran-3,4-diyl diacetate (Intermediate I-D, 100 g, 0.49 mol) in methyl alcohol (1.0 L) was sodium methoxide (2.70 g, 0.05 mol) at 0-10° C., and then the mixture was warmed to 20-25° C. and stirred for 3 hrs. The reaction mixture was adjusted to pH 6-8 with aq. sulfuric acid (6 mol/L) and then filtered. The filter cake was washed with methyl alcohol (50 mL) and the filtrates were concentrated under reduced pressure to afford (3S,4R)-tetrahydro-2H-pyran-3,4-diol (Intermediate I-E, 53.7 g, 92%, crude) as a light yellow oil.

A mixture of (3S,4R)-tetrahydro-2H-pyran-3,4-diol (Intermediate I-E, 30.0 g, crude), (4-fluorophenyl)boronic acid (35.5 g, 0.02 mol), 4-methoxybenzyl chloride (47.8 g, 0.30 mol), potassium carbonate (42.1 g, 0.30 mol) and potassium iodide (42.1 g, 0.25 mol) in acetonitrile (300 mL) was stirred at 70-80° C. for 16 hrs. The reaction mixture was concentrated under reduced pressure, and the residue was dissolved with ethyl acetate (300 mL) and water (300 mL). The aqueous phase was extracted with ethyl acetate (150 mL), and the combined organic phases were washed with brine (15%, 300 mL×2), dried over anhydrous sodium sulfate and concentrated under reduced pressure. The residue was subjected to silica gel column chromatography to afford (3S,4R)-3-((4-methoxybenzyl)oxy)tetrahydro-2H-pyran-4-ol (Intermediate I, 15.2 g) as a yellow oil. ¹H NMR (400 MHz, CDCl₃) δ 7.23-7.14 (m, 2H), 6.87-6.75 (m, 2H), 4.58-4.47 (m, 2H), 3.91 (dt, J=6.4, 3.3 Hz, 1H), 3.77-3.60 (m, 5H), 3.54-3.39 (m, 3H), 2.32 (s, 1H), 1.90-1.83 (m, 1H), 1.78-1.70 (m, 1H).

### Example 1. Synthesis of 4-((4-(((3S,4R)-3-hydroxytetrahydro-2H-pyran-4-yl)oxy)-5-(trifluoromethyl)pyrimidin-2-yl)amino)-N-(methyl-d₃)benzenesulfonamide (E1)

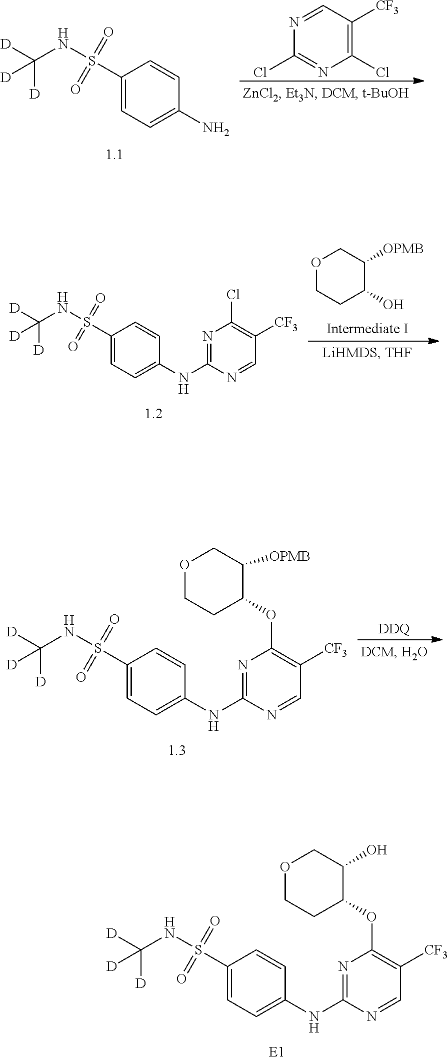

To a solution of 2,4-dichloro-5-(trifluoromethyl)pyrimidine (72.9 g, 0.34 mol) in dichloromethane (290 mL) and tert-butanol (290 mL) was added a solution of zinc chloride (364 mL, 13% in tetrahydrofuran) dropwise at 0-10° C. over 30 mins under nitrogen atmosphere. The mixture was stirred at 0-10° C. for 1 hr followed by addition of 4-amino-N-(methyl-d₃)benzenesulfonamide (1.1, 52.9 g, 0.28 mol) and a solution of triethylamine (36.8 g, 50 mL, 0.36 mol) in dichloromethane/tert-butanol (214 mL, v/v=1/1,) dropwise over 30 mins. The resulting mixture was stirred at 25-30° C. for 40 hrs under nitrogen atmosphere. The reaction mixture was cooled to 0-10° C. and quenched with water (800 mL) slowly. The mixture was warmed to 10-20° C. and stirred for 30 mins, and then it was filtered. The filter cake was washed with dichloromethane (100 mL) and dried in vacuum to afford 4-((4-chloro-5-(trifluoromethyl)pyrimidin-2-yl)amino)-N-(methyl-d₃)benzenesulfonamide as a yellow solid (1.2, 79.0 g, 77%).

To a mixture of 4-((4-chloro-5-(trifluoromethyl)pyrimidin-2-yl)amino)-N-(methyl-d₃)benzenesulfonamide (1.2, 4.08 g, 11.0 mmol) and (3S,4R)-3-((4-methoxybenzyl)oxy)tetrahydro-2H-pyran-4-ol (3.42 g, 14.3 mmol, Intermediate I) in tetrahydrofuran (80 mL) was added lithium hexamethyldisilazide (27.5 mL, 24% in tetrahydrofuran) dropwise under nitrogen atmosphere, and the mixture was stirred at 55-60° C. for 16 hrs. The reaction mixture was cooled to 0-10° C. and quenched with 20% aq. NH₄Cl (16 mL) and water (16 mL). The aqueous phase was extracted with 2-methyltetrahydrofuran (30 mL×2), and the combined organic phases were washed with brine (30 mL) and concentrated under reduced pressure. The residue was subjected to silica gel column chromatography (petroleum ether:ethyl acetate=4:1˜ 1:1) to afford 4-((4-(((3S,4R)-3-((4-methoxybenzyl)oxy)tetrahydro-2H-pyran-4-yl)oxy)-5-(trifluoromethyl)pyrimidin-2-yl)amino)-N-(methyl-d₃)benzenesulfonamide as a yellow solid (1.3, 4.38 g, 70%).

To a solution of 4-((4-(((3S,4R)-3-((4-methoxybenzyl)oxy)tetrahydro-2H-pyran-4-yl)oxy)-5-(trifluoromethyl)pyrimidin-2-yl)amino)-N-(methyl-d₃)benzenesulfonamide (1.3, 3.50 g, 6.12 mmol) in dichloromethane (70 mL) and water (10 mL) was added 2,3-dichloro-5,6-dicyano-1,4-benzoquinone (1.81 g, 7.97 mmol), and the mixture was stirred at 15-20° C. for 5 hrs. The reaction mixture was concentrated in vacuum under 30° C. The residue was redissolved with 2-methyltetrahydrofuran (35 mL) followed by addition of 20% aq. sodium thiosulfate (35 mL), and the mixture was stirred at 15-20° C. for 30 mins. The organic phase was washed with 5% aq. sodium bicarbonate (35 mL×2) and brine (35 mL), and then it was concentrated under reduced pressure. The residue was subjected to silica gel column chromatography (heptane:ethyl acetate=1:1˜ 1:2) to afford 4-((4-(((3S,4R)-3-hydroxytetrahydro-2H-pyran-4-yl)oxy)-5-(trifluoromethyl)pyrimidin-2-yl)amino)-N-(methyl-d₃)benzenesulfonamide (E1, 3.36 g, 83%). LC-MS (ESI):m/z 452.2 [M+H]⁺. ¹H NMR (400 MHz, DMSO-d₆) δ 10.50 (s, 1H), 8.60 (s, 1H), 7.92 (d, J=8.8 Hz, 2H), 7.73 (d, J=8.7 Hz, 2H), 7.27 (s, 1H), 5.62-5.56 (m, 1H), 5.05 (d, J=4.9 Hz, 1H), 3.94-3.85 (m, 1H), 3.69-3.49 (m, 4H), 2.10-1.81 (m, 2H).

LC-MS and ¹H NMR for additional exemplary compounds which were prepared with chiral starting material/intermediate, using similar synthetic methodology (with different starting material, intermediate, or stereoisomer) and routes of this Illustration are provided in Table A, below.

TABLE A
Exemplary Characterization of Additional Compounds of the Present disclosure

| Compound | | |
|---|---|---|
| No. | Structure | LC-MS; ¹H NMR (ppm) |
| E2 | 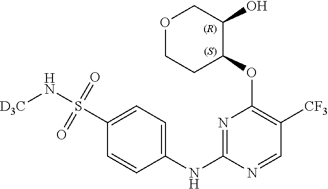 | LC-MS (ESI): m/z 452.2 [M + H]⁺; ¹H NMR (400 MHz, DMSO-d₆) δ 10.50 (s, 1H), 8.61 (s, 1H), 7.93 (d, J = 8.8 Hz, 2H), 7.74 (d, J = 8.8 Hz, 2H), 7.27 (s, 1H), 5.68-5.51 (m, 1H), 5.06 (s, 1H), 3.90 (s, 1H), 3.67-3.48 (m, 4H), 2.01 (dd, J = 6.2, 3.7 Hz, 1H), 1.96-1.82 (m, 1H). |
| E3 | 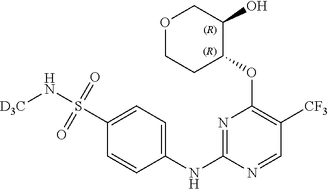 | LC-MS (ESI): m/z 452.2 [M + H]⁺; ¹H NMR (400 MHz, DMSO-d₆) δ 10.54 (s, 1H), 8.62 (s, 1H), 7.94 (d, J = 8.8 Hz, 2H), 7.74 (d, J = 8.8 Hz, 2H), 7.28 (s, 1H), 5.28 (t, J = 8.9 Hz, 2H), 3.81 (dt, J = 10.8, 5.4 Hz, 2H), 3.68-3.57 (m, 1H), 3.53-3.43 (m, 1H), 3.28 (dd, J = 11.4, 8.0 Hz, 1H), 2.28-2.14 (m, 1H), 1.70- 1.55 (m, 1H). |
| E4 | 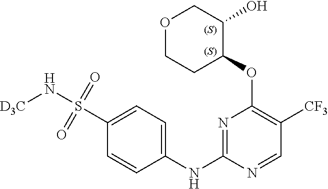 | LC-MS (ESI): m/z 452.2 [M + H]⁺; ¹H NMR (400 MHz, DMSO-d₆) δ 10.54 (s, 1H), 8.62 (s, 1H), 7.94 (d, J = 8.7 Hz, 2H), 7.74 (d, J = 8.7 Hz, 2H), 7.28 (s, 1H), 5.28 (t, J = 8.5 Hz, 2H), 3.88-3.74 (m, 2H), 3.69-3.56 (m, 1H), 3.48 (t, J = 9.3 Hz, 1H), 3.28 (dd, J = 11.3, 8.1 Hz, 1H), 2.22 (dd, J = 9.4, 3.9 Hz, 1H), 1.71- 1.55 (m, 1H). |
| E5 | 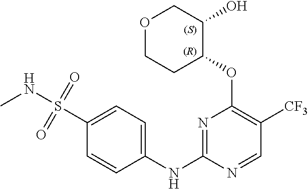 | LC-MS (ESI): m/z 449.2 [M + H]⁺; ¹H NMR (400 MHz, DMSO-d₆) δ 10.50 (s, 1H), 8.60 (s, 1H), 7.94 (d, J = 8.8 Hz, 2H), 7.75 (d, J = 8.8 Hz, 2H), 7.30 (s, 1H), 5.70-5.50 (m, 1H), 5.06 (d, J = 3.9 Hz, 1H), 3.91 (d, J = 3.3 Hz, 1H), 3.69- 3.50 (m, 4H), 2.42 (s, 3H), 2.01 (dd, J = 6.2, 3.7 Hz, 1H), 1.91 (dd, J = 7.0, 2.9 Hz, 1H). |
| E6 | 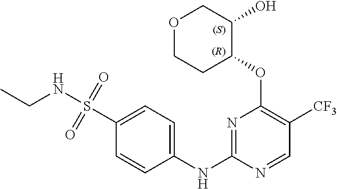 | LC-MS (ESI): m/z 463.1 [M + H]⁺; ¹H NMR (400 MHz, DMSO-d₆) δ 10.50 (s, 1H), 8.60 (s, 1H), 7.92 (d, J = 8.8 Hz, 2H), 7.75 (d, J = 8.8 Hz, 2H), 7.41 (t, J = 5.7 Hz, 1H), 5.66-5.52 (m, 1H), 5.06 (d, J = 4.9 Hz, 1H), 3.90 (td, J = 7.5, 4.2 Hz, 1H), 3.67-3.50 (m, 4H), 2.83-2.71 (m, 2H), 2.08-1.97 (m, 1H), 1.90 (dtd, J = 9.5, 6.7, 3.0 Hz, 1H), 0.98 (t, J = 7.2 Hz, 3H). |
| E7 | 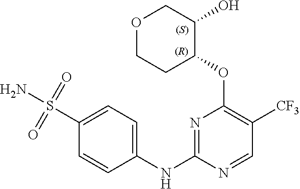 | LC-MS (ESI): m/z 435.2 [M + H]⁺; ¹H NMR (400 MHz, DMSO-d₆) δ 10.46 (s, 1H), 8.60 (s, 1H), 7.88 (d, J = 8.8 Hz, 2H), 7.78 (d, J = 8.9 Hz, 2H), 7.25 (s, 2H), 5.66-5.52 (m, 1H), 5.07 (d, J = 4.7 Hz, 1H), 3.90 (d, J = 3.7 Hz, 1H), 3.67- 3.52 (m, 4H), 2.09-1.96 (m, 1H), 1.90 (dd, J = 7.0, 2.9 Hz, 1H). |

Biological activity for representative compounds of the present disclosure were tested according to those described in Biological Example 1 in PCT/CN2021/133429, published as WO2022/111621 on Jun. 2, 2022. Exemplary results are presented as calculated IC₅₀ values and are presented in Table 2 below. In Table 2, “A” represents a calculated IC₅₀ value of less than 10 nM; “B” represents a calculated IC₅₀ value of greater than or equal to 10 nM and less than 100 nM; “C” represents a calculated IC₅₀ value of greater than or equal to 100 nM and less than 1 μM; and “D” represents a calculated IC₅₀ value of 1 μM or greater.

TABLE 2
Selected in vitro data on different CDKs

| | | | | | CDK7/ | |
|---|---|---|---|---|---|---|
| | CDK1/ | CDK2/ | CDK4/ | CDK6/ | Cyclin H/ | CDK9/ |
| Compound | Cyclin B1 | Cyclin E1 | Cyclin D1 | Cyclin D1 | MAT1 IC50 | Cyclin T1 |
| number | IC50(nM) | IC50 (nM) | IC50 (nM) | IC50 (nM) | (nM) | IC50 (nM) |
| E1 | B | A | C | C | C | C |
| E2 | C | B | D | D | D | C |
| E3 | B | A | C | D | C | C |
| E4 | C | B | D | D | D | D |
| E7 | C | A | C | C | D | D |

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

1-16. (canceled)

17. A compound selected from: 4-((4-(((3S,4R)-3-((4-methoxybenzyl)oxy)tetrahydro-2H-pyran-4-yl)oxy)-5-(trifluoromethyl)pyrimidin-2-yl)amino)benzenesulfonamide; 4-((4-(((3S,4R)-3-((4-methoxybenzyl)oxy)tetrahydro-2H-pyran-4-yl)oxy)-5-(trifluoromethyl)pyrimidin-2-yl)amino)-N-methylbenzenesulfonamide; N-ethyl-4-((4-(((3S,4R)-3-((4-methoxybenzyl)oxy)tetrahydro-2H-pyran-4-yl)oxy)-5-(trifluoromethyl)pyrimidin-2-yl)amino)benzenesulfonamide; 4-((4-(((3S,4R)-3-((4-methoxybenzyl)oxy)tetrahydro-2H-pyran-4-yl)oxy)-5-(trifluoromethyl)pyrimidin-2-yl)amino)-N-(methyl-d3)benzenesulfonamide; 4-((4-(((3R,4R)-3-((4-methoxybenzyl)oxy)tetrahydro-2H-pyran-4-yl)oxy)-5-(trifluoromethyl)pyrimidin-2-yl)amino)-N-(methyl-d3)benzenesulfonamide; 4-((4-(((3S,4S)-3-((4-methoxybenzyl)oxy)tetrahydro-2H-pyran-4-yl)oxy)-5-(trifluoromethyl)pyrimidin-2-yl)amino)-N-(methyl-d3)benzenesulfonamide; and 4-((4-(((3R,4S)-3-((4-methoxybenzyl)oxy)tetrahydro-2H-pyran-4-yl)oxy)-5-(trifluoromethyl)pyrimidin-2-yl)amino)-N-(methyl-d3)benzenesulfonamide.

18. A compound, or a pharmaceutically acceptable salt thereof, wherein the compound is selected from the following:

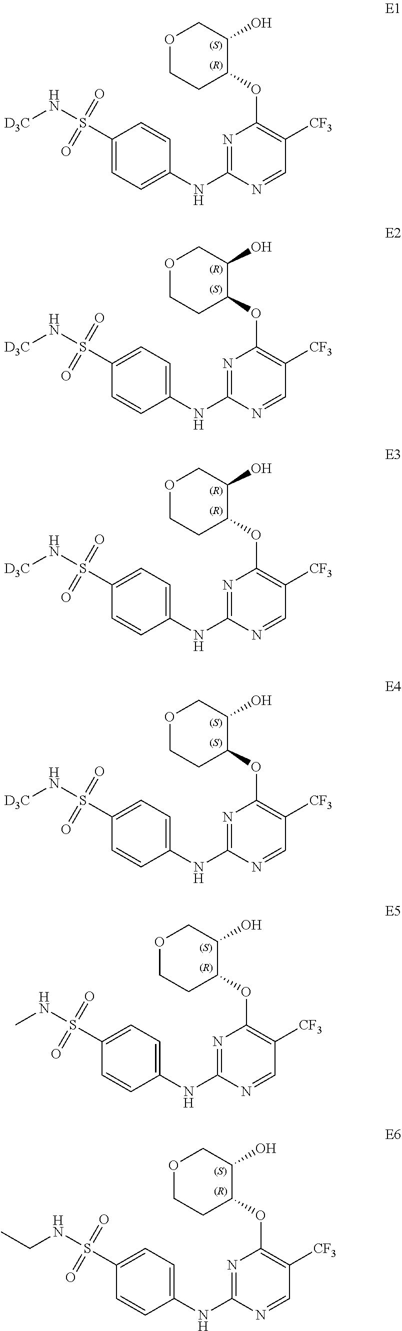

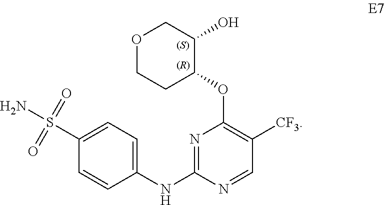

19. A pharmaceutical composition comprising the compound of claim 18, or pharmaceutically acceptable salt thereof, and a pharmaceutically acceptable excipient.

20. A method of treating cancer in a subject in need thereof, the method comprising administering to the subject a therapeutically effective amount of the compound, or pharmaceutically acceptable salt thereof, of claim 18.

21. The method of claim 20, wherein the cancer is breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, or thyroid cancer.

22. The method of claim 20, wherein the cancer is breast cancer selected from ER-positive/HR-positive, HER2-negative breast cancer, ER-positive/HR-positive, HER2-positive breast cancer, triple negative breast cancer, or inflammatory breast cancer.

23. The method of claim 20, wherein the cancer is breast cancer selected from endocrine resistant breast cancer, trastuzumab resistant breast cancer, or breast cancer demonstrating primary or acquired resistance to CDK4/CDK6 inhibition.

24. The method of claim 20, wherein the cancer is advanced or metastatic breast cancer.

25. The method of claim 20, wherein the cancer is ovarian cancer.

26. The method of claim 20, wherein the cancer is characterized by an amplification or overexpression of cyclin E1 and/or cyclin E2.

27. A method of producing (3S,4R)-3-((4-methoxybenzyl)oxy)tetrahydro-2H-pyran-4-ol, comprising reacting (3S,4R)-tetrahydro-2H-pyran-3,4-diol with (4-fluorophenyl)boronic acid and 4-methoxybenzyl chloride.

28. The method of claim 27, further comprising reacting (3S,4R)-tetrahydro-2H-pyran-3,4-diyl diacetate with sodium methoxide to produce the (3S,4R)-tetrahydro-2H-pyran-3,4-diol.

29. The method of claim 28, further comprising reacting (3S,4R)-3,4-dihydro-2H-pyran-3,4-diyl diacetate with a palladium catalyst and hydrogen to produce the (3S,4R)-tetrahydro-2H-pyran-3,4-diyl diacetate.

30. The method of claim 29, further comprising reacting (3R,4S,5S)-2-bromotetrahydro-2H-pyran-3,4,5-triyl triacetate with sodium acetate and a mixture of copper acetate, acetic acid, and zinc dust to produce the (3S,4R)-3,4-dihydro-2H-pyran-3,4-diyl diacetate.

31. The method of claim 30, further comprising reacting (3R,4S,5S)-tetrahydro-2H-pyran-2,3,4,5-tetraol with 4-dimethylaminopyridine and acetic anhydride to produce the (3R,4S,5S)-2-bromotetrahydro-2H-pyran-3,4,5-triyl triacetate.

32. The method of claim 31, wherein the reacting of the (3S,4R)-tetrahydro-2H-pyran-3,4-diol, (4-fluorophenyl)boronic acid, and 4-methoxybenzyl chloride is performed in the presence of potassium carbonate and potassium iodide.

33. A method of producing 4-((4-(((3S,4R)-3-hydroxytetrahydro-2H-pyran-4-yl)oxy)-5-(trifluoromethyl)pyrimidin-2-yl)amino)-N-(methyl-d₃)benzenesulfonamide, comprising reacting 4-((4-(((3S,4R)-3-((4-methoxybenzyl)oxy)tetrahydro-2H-pyran-4-yl)oxy)-5-(trifluoromethyl)pyrimidin-2-yl)amino)-N-(methyl-d₃)benzenesulfonamide with 2,3-dichloro-5,6-dicyano-1,4-benzoquinone.

34. The method of claim 33, further comprising reacting 4-((4-chloro-5-(trifluoromethyl)pyrimidin-2-yl)amino)-N-(methyl-d₃)benzenesulfonamide with (3S,4R)-3-((4-methoxybenzyl)oxy)tetrahydro-2H-pyran-4-ol and lithium hexamethyldisilazide to produce the 4-((4-(((3S,4R)-3-((4-methoxybenzyl)oxy)tetrahydro-2H-pyran-4-yl)oxy)-5-(trifluoromethyl)pyrimidin-2-yl)amino)-N-(methyl-d₃)benzenesulfonamide.

35. The method of claim 34, further comprising reacting 4-amino-N-(methyl-d₃)benzenesulfonamide with 2,4-dichloro-5-(trifluoromethyl)pyrimidine, zinc chloride and triethylamine to produce 4-((4-chloro-5-(trifluoromethyl)pyrimidin-2-yl)amino)-N-(methyl-d₃)benzenesulfonamide.

36. A compound which is 4-((4-(((3S,4R)-3-((4-methoxybenzyl)oxy)tetrahydro-2H-pyran-4-yl)oxy)-5-(trifluoromethyl)pyrimidin-2-yl)amino)-N-(methyl-d₃)benzenesulfonamide.

37. A compound which is 4-((4-chloro-5-(trifluoromethyl)pyrimidin-2-yl)amino)-N-(methyl-d₃)benzenesulfonamide.

38. A compound which is (3S,4R)-3-((4-methoxybenzyl)oxy)tetrahydro-2H-pyran-4-ol.
